(19) United States
(12) Patent Application Publication
(10) Pub. No.: US 2019/0201812 A1
(43) Pub. Date: Jul. 4, 2019

(54) Functionalized Polyamide Stationary Phase for Chromatography and Microwave Assisted Formation Thereof

(71) Applicant: CLEMSON UNIVERSITY, CLEMSON, SC (US)

(72) Inventors: R. Kenneth MARCUS, CLEMSON, SC (US); LIUWEI JIANG, CENTRAL, SC (US)

(21) Appl. No.: 16/311,750

(22) PCT Filed: Jun. 20, 2017
(86) PCT No.: PCT/US2017/038268

§ 371 Date: Dec. 20, 2018

Related U.S. Application Data
(60) Provisional application No. 62/352,247, filed on Jun. 20, 2016.

(51) Int. Cl. B01D 15/20; B01J 20/26; B01J 20/286; B01J 20/32; B01J 20/30; B01J 20/28

(52) U.S. Cl. CPC B01D 15/20; B01J 20/264; B01J 20/286; B01J 20/3212; B01D 15/361; B01J 20/3078; B01J 20/28023; B01J 2220/58; B01J 20/3278

(57) ABSTRACT

Chromatography devices and methods for forming and using the devices are described. The devices include a polyimide-based support phase and a polymer grafted to a surface of the polyimide-based support phase. A microwave-assisted graft polymerization protocol is described to form the polymer at the surface of the support phase. Devices can be utilized in high-efficiency separation of macromolecules such as proteins.

### CROSS-REFERENCE TO RELATED APPLICATION

This application claims filing benefit of U.S. Provisional Patent Application Ser. No. 62/352,247 having a filing date of Jun. 20, 2016 entitled “Use of Microwave Energy to Affect Changes in Chemical Functionality of Nylon Surfaces,” which is incorporated herein by reference for all purposes.

### STATEMENT REGARDING FEDERALLY SPONSORED RESEARCH OR DEVELOPMENT

This invention was made with government support under grant nos. CHE-1307078 and 1608663 awarded by National Science Foundation. The government has certain rights in the invention.

### BACKGROUND

Traditional liquid chromatography stationary phases are composed of highly porous, micro-sphere packed-bed support phases. While the majority of supports are silica-based, polymeric materials are finding wider application due to their chemical robustness. There also exists a rich tool box of surface modification chemistries to improve function of the basic chromatography support materials. Successful surface modification strategies have been pursued on polymeric-based supports to affect greater efficiency/selectivity while retaining the basic hydrodynamic properties of the polymers. For example, simple surface amination of polyethylene terephthalate (PET) support materials has allowed creation of a biotinylated surface as stationary phase for affinity separations, and the creation of polymeric polyethyleneimine (PEI) phases has been developed for weak anion exchange, for instance in protein separations.

In recent years, macromolecular therapeutics and in particular protein-based therapeutics have played increasingly important roles in the pharmaceutical industry. The manufacture of proteinaceous therapeutics involves two major operations, upstream processing (e.g., production via cell culturing/fermentation) and downstream processing (e.g., purification/recovery). While both upstream and downstream processing have been improved over the last two decades, downstream processing continues to be the rate-limiting step in protein-based drug production. Chromatographic separation in particular causes a bottle-neck in the downstream processing due to its high costs and time consumption.

In terms of the ability to mitigate any decomposition/deactivation of biologically active materials such as proteins, ion exchange-based (IEX) separations are extremely attractive in downstream macromolecular therapeutic processing. Unfortunately, hydrophobic interactions between such materials, e.g., polypeptides and typical non-polar chromatography support/stationary phases lead to separations that are mixed-mode in nature. Hydrophobic interactions can cause proteins to de-nature during separations and can lead to low product recoveries and peak tailing in chromatograms. As such, hydrophilic supports are preferred in IEX protein separations.

Highly hydrophilic polyimide-based supports (e.g., nylon 6) have been used in IEX protein separations based on the acid/base character of the natural carboxylic acid and primary amine end groups of the polyamides. Unfortunately, the low density of these ion-exchange ligands limits the performance of the native materials. Additional surface modifications on the native nylon could improve the chromatographic properties of this phase. Different methods have been reported in the literature for the modification of nylon materials used in non-chromatography applications. Some of these modification methods target activation of the amide groups, unfortunately resulting in cleavage of the amide bond and causing inevitable physical damage to the nylon bulk structure. Other methods that require energy input (e.g., UV treatment, conventional heating, plasma beam treatment) can render the approach either not conducive to large-scale processing (due to, e.g., cost and/or incomplete surface access) and/or lead to degradation of the nylon-based support material. Such modification approaches are thus highly problematic in development of polyamide-based chromatography support and stationary phase materials.

Separation of macromolecules is also problematic due to high mass transfer resistance as the large target species diffuse through the pores of particulate-packed columns. New forms for chromatographic materials have been proposed for macromolecule separations such as fiber-based materials and superficially porous silica microspheres composed of non-porous cores and thin porous outer shells (0.1-1 μm). Such columns have been shown to be capable of separation of macromolecules at relatively high mobile phase velocities. Monolithic columns that provide high mass transfer efficiencies along with the chemical robustness desirable for protein IEC separations have also attracted attention for macromolecule separations.

While the above describes improvements in the art, there is a continuous interest in the development of support phases for chromatography applications that can provide high-throughput and cost-effective separations. Support phases that could be utilized in macromolecule separations could be of particular benefit.

### SUMMARY

According to one embodiment, disclosed is a separation apparatus that includes a fluid conduit having a first end and a second end that is disposed opposite the first end. The separation apparatus also includes a support phase disposed within the conduit between the first end and the second end. The support phase includes a polymer grafted at a surface of the support phase, the polymer including a chromatography functionality (e.g., an ion exchange functionality) as a stationary phase for a separation protocol. The support phase is formed of a polymeric composition that includes a polyamide, e.g., a nylon.

In one embodiment, the support phase can be in the form of a fiber, and in one particular embodiment, in the form of a capillary-channeled polymeric (C-CP) fiber. Accordingly, in one embodiment, disclosed is a separation device that includes a C-CP fiber formed of a polyamide-containing polymeric composition that in turn includes a polymer grafted at a surface of the C-CP fiber, the polymer including a chromatography functionality.

Also disclosed is a method for forming a separation apparatus. A method can include contacting a support phase with a solution that includes polymerizable monomers (or oligomers) and a polymerization initiator. More specifically, the support phase can be formed of a polymeric composition that includes a polyamide. The method can also include contacting the solution with energy in the microwave spectrum and thereby encouraging radical polymerization of the monomers at the surface of the support phase. The surface grafted polymerization product including a chromatography functionality.

According to another embodiment, disclosed is a method for separating a species from a fluid. The method can include moving a fluid through a conduit that contains a support phase, the support phase including a polymeric structure (e.g., a nylon-based capillary-channeled fiber) that includes a polymer grafted at a surface of the polymeric structure, the polymer including a chromatography functionality as a stationary phase for a separation protocol. Upon moving the fluid through the conduit, the species of interest can preferentially adhere to the stationary phase via interaction with the chromatography functionality (e.g., ion exchange). In one particular embodiment, the species of interest can be a macromolecule, e.g., a protein or proteinaceous therapeutic.

Additional objects and advantages of the invention will be set forth in part in the description which follows, and in part will be obvious from the description, or may be learned by practice of the invention. The objects and advantages of the invention may be realized and attained by means of the instrumentalities and combinations particularly pointed out in the appended claims.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 schematically illustrates a separation column as described herein.

FIG. 2 is a perspective view of an end of a single capillary-channeled polymer (C-CP) fiber.

FIG. 3 presents a cross-sectional view of a plurality of C-CP fibers packed together.

FIG. 4 illustrates several different examples of C-CP fibers and spinnerets thereof.

FIG. 5 schematically illustrates one method for forming a separation column.

FIG. 6 provides an ATR-FTIR spectrum of the native and modified nylon C-CP fibers.

FIG. 7 provides several SEM images of native nylon C-CP fibers (left) and modified nylon-COON C-CP fibers (right).

FIG. 8 presents lysozyme loading breakthrough curves on a nylon-COON C-CP fiber column at constant mobile phase linear velocity and various protein loading concentrations. The breakthrough curves are plotted on the time basis.

FIG. 9 presents lysozyme loading breakthrough curves on a nylon-COON C-CP fiber column at constant protein loading concentration and various mobile phase linear velocities.

FIG. 10 provides chromatographs of 10 continuous lysozyme loading/elution cycles on a nylon-COON C-CP fiber column without column regeneration in between.

FIG. 11 provides chromatographs of lysozyme loading/elution on 5 replicate nylon-COON C-CP fiber columns that were prepared at different times.

FIG. 12 illustrates results of separations of (1) myoglobin, (2) α-chymotrypsinogen A, (3) cytochrome C and (4) lysozyme on native and nylon-COON C-CP fiber columns at different linear velocities.

FIG. 13 is a table presenting surface characteristics reflecting the conditions for Example 2.

FIG. 14 presents ATR-FTIR spectra of the native and modified nylon 6 C-CP fibers.

FIG. 15 presents SEM images of native nylon 6 C-CP fibers and modified nylon-SO₃H C-CP fibers.

FIG. 16 presents column backpressure as a function of mobile phase linear velocity for separations columns as described herein.

FIG. 17 presents the effect of lysozyme concentration on the dynamic loading capacity for native nylon and nylon-SO₃H fiber columns. Note that errors bars for duplicate determinations are within the many of the data symbols.

FIG. 18 presents chromatograms of the separation of myoglobin, α-chymotrypsinogen A and lysozyme (left to right) on different nylon-SO₃H columns.

FIG. 19 presents chromatograms for the separation of myoglobin, α-chymotrypsinogen A and lysozyme (left to right) on a nylon-SO₃H column at four different flow rates.

FIG. 20 presents the effect of gradient time and mobile phase linear velocity on (a) separation resolution of α-chymotrypsinogen A and lysozyme (b) peak capacity on a nylon-SO₃H column.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, disclosed are separation devices that include polyamide-based support materials and methods for forming and using the devices. More specifically, the separation devices include a polyamide-based support phase and a polymer grafted to a surface of the polyamide-based support phase that provides a ligand as a stationary phase for a separation protocol. A formation method can include a microwave-assisted grafting polymerization protocol to form the polymer at the surface of the support phase. The microwave-assisted modification is a versatile method that can introduce any of a large variety of functional ligands onto the support phase phase and can thus greatly expand and improve the application of polyamide support phases for separations, and in one particular embodiment, for macromolecular separations. Formation of the support materials as described can provide high density of stationary phase functionality over the entire surface of support materials, even when using support materials with non-uniform shapes, and can do so without undesirable degradation of the polyamide-based support materials.

Specific applications for the devices include but are not limited to analytical separations such as liquid chromatography (HPLC, cap-LC); prep-scale separations; micro-scale separations; single fiber separations; extraction of selected organic molecules/ions from solution; purification of liquid streams (process waste, drinking water, pure solvents); selective extraction of cell matter and bacteria from growth media; and immobilization of cell matter and bacteria. Potential applications can include analytical instrumentation; specialty chemicals; and pharmaceutical applications. Demand for the product can be based on its advantages in attaining high throughput and productivity.

Disclosed materials can be utilized in certain embodiments in separation of macromolecular species, and in one particular embodiment in proteinaceous separations (i.e., proteins or biologically active polypeptides) via ion exchange, including strong cation exchange (SCX) and weak cation exchange (WCX) separations, without sacrificing the desirable nature of the hydrophilic surface or the column hydrodynamics of the polyamide-based support medium. For instance, fiber-based packed columns of the disclosed materials can exhibit significantly increased dynamic binding capacity as compared to native polyamide support materials. While much of this disclosure is directed to ion exchange chromatography methods and functionalities, the materials and methods are in no way limited to ion exchange materials or methods, and any chromatography methodology and functionality is encompassed herein. For example, disclosed separation devices can be modified to include chromatography functionality suitable for ion exchange chromatography (cation or anion, strong or weak), hydrophilic interaction chromatography, molecular recognition-based affinity chromatography, immobilized-metal affinity chromatography, metal ion chelation (e.g., separations and extractions), etc. Additionally, other sorts of chromatography functionality can be affected by coupling so-called linker molecules which allow further attachments of functional elements based on ester chemistry, amine chemistry, click reaction chemistry, epoxide chemistry, etc.

As utilized herein the term “macromolecular” generally refers to a molecule having a number average molecular weight of about 1,000 or greater, or 5,000 or greater in some embodiments.

The term, “proteinaceous” generally refers to a polypeptide having a biological activity and can include complete proteins or functional fragments thereof as well as synthetic polymeric materials formed of natural and/or synthetic amino acid residues bonded to one another via a peptide linkage.

The polyamide-based support phase can have any physical shape and size conducive to fluid and solute movement. For instance, the polyamide-based support phase can be in the form of beads of any shape (e.g., rod, sphere, plate, etc.) and can be either porous or non-porous. In one embodiment the support phase can be in the form of fibers and in one particular embodiment, the support phase can include polymer fibers having a non-circular cross-sectional geometry. The cross-sectional geometry of the fibers can arise from open capillary channels extending continuously along the fiber surface over the entire length of the fiber (i.e., C-CP fibers). Use of surface-channeled fibers (also referred to as C-CP fibers throughout this disclosure) can allow for a wide range of liquid flow rates with very low backing pressures. In one embodiment, a single surface channeled fiber can be used in single fiber separations. For example a column structure can take the form of a single fiber in-laid in a micro-machined device. In another embodiment, bundles of fibers having a channeled cross-sectional geometry and carrying the stationary phase materials can be packed into columns.

C-CP fibers can be beneficial in chromatographic separations as they can provide for high contact area and high linear velocity with low back pressure. For instance, capillary-channeled fibers can provide relatively small interstitial fractions within the column (i.e., the interstitial volume per unit volume of the packed column), for instance about 1.0 or less, about 0.75 or less, or about 0.65 or less in some embodiments and high fiber density such as about 4 mg/cm³ or greater, or about 5 mg/cm³ or greater in some embodiments. Meanwhile, a column of packed capillary-channeled fibers can be operated at a linear velocity of about 25 mm/sec or greater, for instance about 50 mm/sec or greater or about 100 mm/sec in some embodiments, with a back pressure of about 2000 psi or less. C-CP fibers can be similar to those disclosed in U.S. Pat. Nos. 7,740,763; 7,374,673, and 7,261,813; incorporated herein by reference.

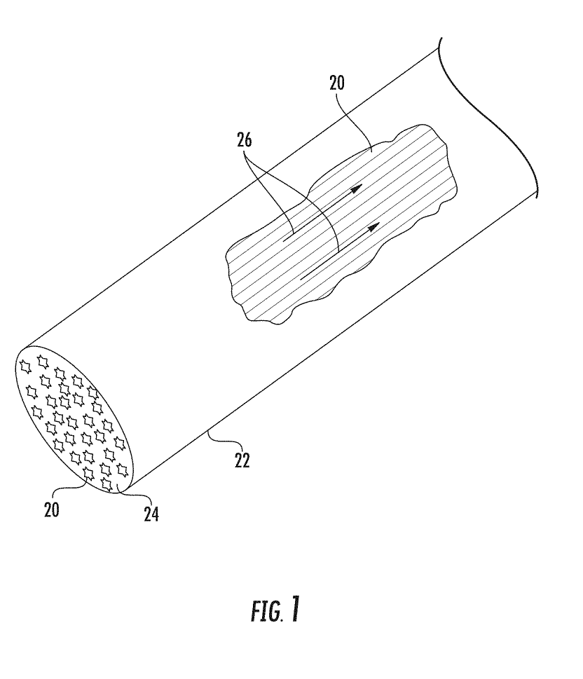

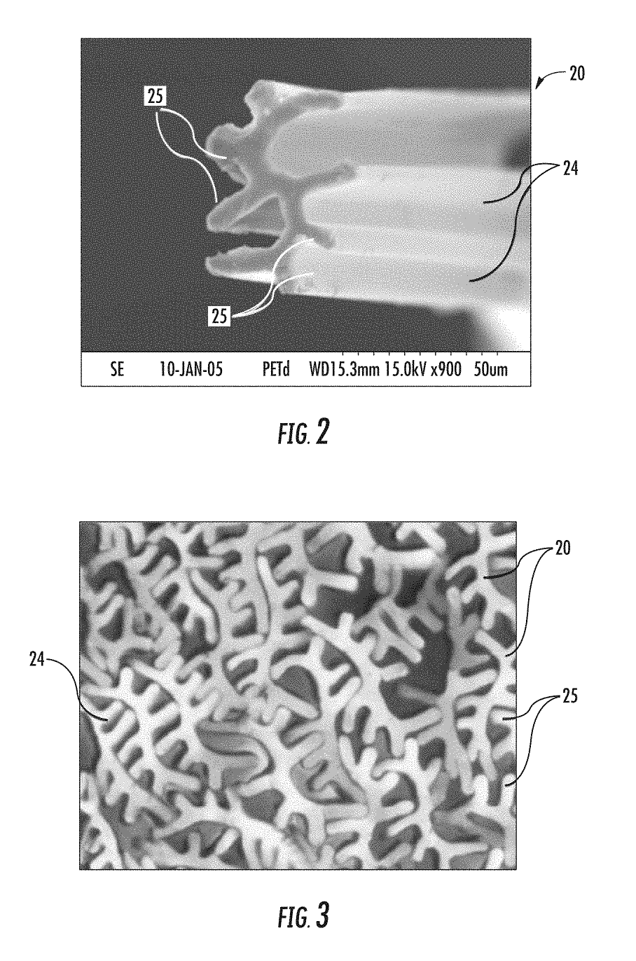

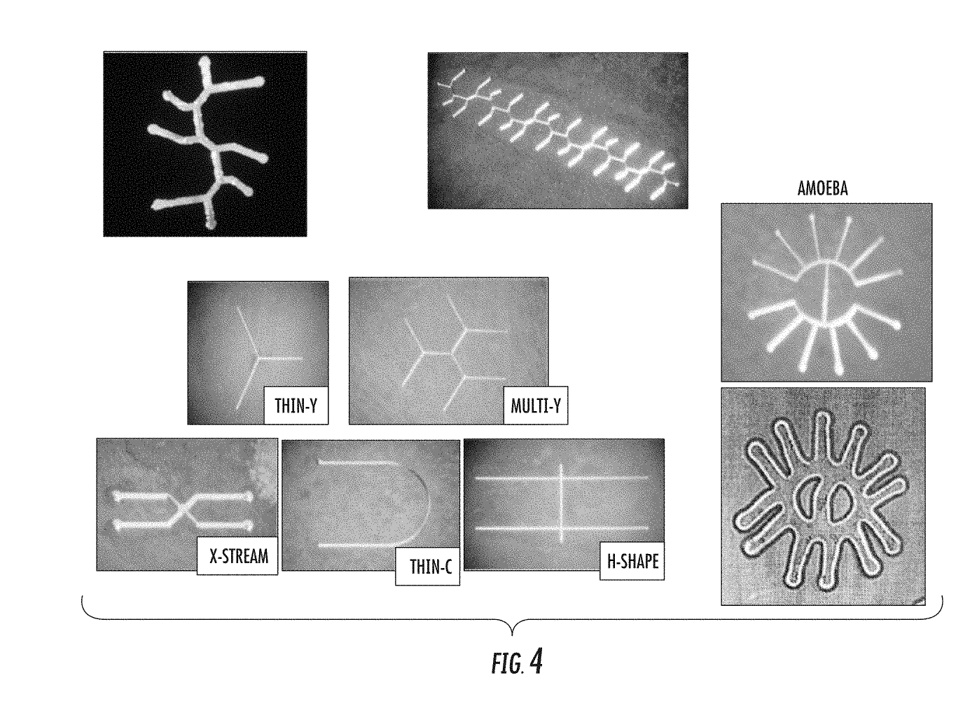

Referring to FIG. 1, a perspective view of a plurality of C-CP fibers 20 are shown packed into a casing 22. FIG. 2 illustrates a single fiber 20 in perspective view showing the individual capillary channels 24 that are open at the fiber surface and that extend along the length of the fiber 20, and FIG. 3 presents a cross-sectional view of a plurality of C-CP fibers packed together, as in a casing for a separation device. As shown in FIGS. 1-3, each fiber strand 20 has a plurality of co-linear capillaries or channels 24 extending the entire length of the exterior surface of the fiber 20. Each capillary 24 is defined by a pair of opposed walls 25 that extend generally radially and longitudinally and form part of the exterior surface of the fiber 20 (FIG. 2, FIG. 3). Desirably, these walls 25 and capillaries 24 defined thereby extend down the entire length of the fiber 20 parallel to the longitudinal axis of the fiber 20 and are nominally co-linear on each fiber 20. This produces de facto substantially the same co-linear capillaries 24 along the entire length of the casing 22 (FIG. 1).

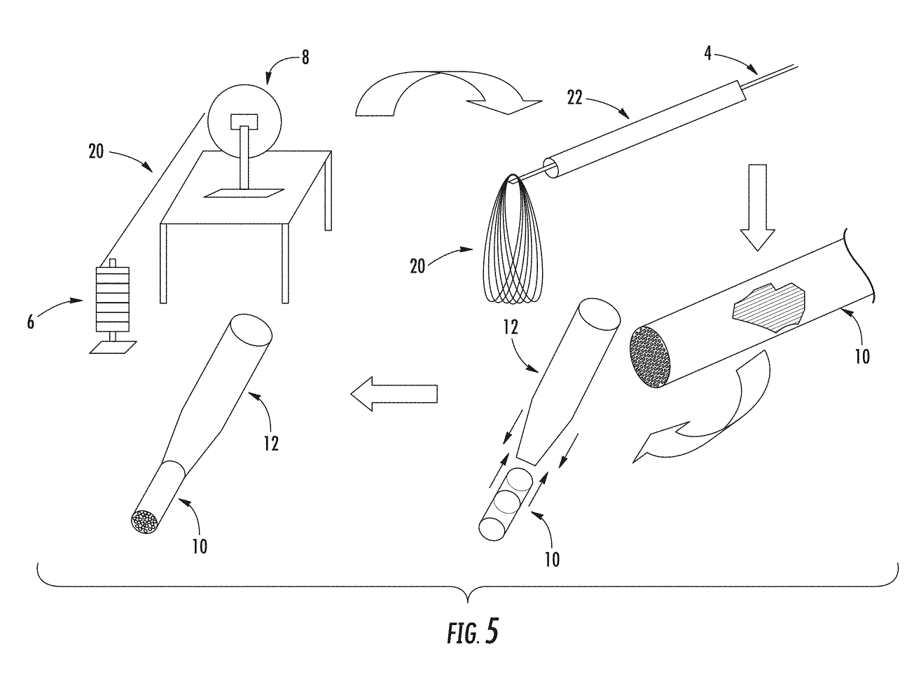

It should be understood that the particular shapes of the C-CP fibers illustrated in FIGS. 1-3 are not a requirement of the present disclosure. In particular, the number and/or cross-sectional shape of the capillaries as well as the overall shape of the C-CP fibers can vary from that shown in the figures. For instance, the depth of a single capillary on a fiber, i.e., the radial height of walls 25 on FIG. 2, can range, for instance, between about 1 μm and about 20 μm. FIG. 4 presents several different variants of C-CP fibers as are encompassed herein.

In one embodiment, the capillaries 24 can be configured to wrap around the length of the fiber 20 in a helical fashion. In one embodiment, substantially all of the capillaries 24 can be nominally co-linear on each fiber 20. As such, substantially all of the capillaries 24 of a plurality of fibers 20 can follow a helix pattern that has a similar pitch. The pitch is the number of complete turns of a single capillary 24 around the circumference of the fiber 20 per unit of length of the fiber 20. This also can produce de facto substantially the same co-linear capillaries 24 along the entire length of the casing 22.

Additionally, in the course of packing the fibers 20 into a bundle that lays along the entire length of the casing 22, whether the individual fibers have purely linear capillaries 24 or helical ones, it is possible that one or more, even all, of the fibers 20 in the bundle will rotate about its/their own axis or the axis of the casing 22 over the entire length of the column. In other words, the capillary-channeled fibers 20 may twist as they lay within the casing 22. Accordingly, the capillaries 24 and walls 25 also may twist somewhat. When collinearly packed together, as in column formats as in FIG. 3, the C-CP fibers can interdigitate to create parallel channels, for instance with average separation distances of about 1 μm to about 5 μm.

The support phase structures (e.g., C-CP fibers) can be formed to a suitable size and shape for the desired separation protocol. For example, fibers can be formed to a desired size and shape to promote capillary flow of a liquid with a predetermined viscosity through a casing. For instance, the nominal diameter of a fibrous solid phase structure (e.g., the diameter of the footprint encompassing the surface capillaries in the case of a C-CP fiber) can range from about 10 μm to about 80 μm, or from about 35 μm to about 50 μm, with channel widths (i.e., the distance from one opposing wall to another at the outermost edge of the channel) of about 25 μm or less, for instance from about 1 μm to about 20 μm in some embodiments. C-CP fibers can possess a specific surface area on par with monolithic materials, for instance from about 2 m² g⁻¹ to about 5 m² g⁻¹.

While much of this disclosure is directed to separation materials utilizing as a support phase one or more C-CP fibers, it should be understood that the support phase is not limited to C-CP fibers, and other geometries are contemplated herein, including, without limitation, circular fibers, hollow fibers, solid and/or porous beads of any desired geometry, monolithic support phases, including porous and channeled monoliths, membranes and filters in the form of films or sheets having a large cross sectional area on a face and a relatively small cross sectional dimension from one side to the other, and so forth.

The polymeric support phase can be formed of a polymeric composition that includes a polyamide (i.e., a nylon). Polyamides for use as described herein can encompass any long-chain with recurring amide groups, as is generally known in the art, including both aliphatic and semi-aromatic polyamides formed via polymerization reaction of lactams, acid/amines, or stoichiometric mixtures of diamines and diacids to provide the desired repeating units linked by peptide bonds. The polyamide can include amide groups in the chain and primary amine and carboxylic acid end groups. The amine and carboxylic acid end groups in the native polymer can provide the electrostatic interaction sites for ion exchange, while the amide moiety enhances the hydrophilicity of the nylon surface, reducing the hydrophobic interactions with components in the mobile phase (e.g., proteins).

The polymeric composition can include additional components as are known in the art, e.g., additives such as clarifiers, nucleating agents, stabilizing agents, other polymers or polymer components, and the like, in conjunction with the polyamide polymer.

There are many different fabrication approaches that can be utilized to form the polyamide-based support phase structures. For instance, thermoplastic polyamide-based C-CP fibers are amenable to formation via extrusion or any other melt processing formation technique.

The characteristics of polyamide-based C-CP fibers can present a number of advantages for macromolecule separations. For instance, the polyamide fibers can be formed so as to be virtually non-porous such that species targeted by the separation protocol are not retained within pores of the support phase. This can eliminate C-term band broadening common to macromolecule chromatography on porous phases.

Additionally, the channel geometry of the C-CP fibers can provide very efficient solvent transport with low flow resistance, allowing for high linear velocities and low back pressures. Moreover, the polyamide polymer can provide a route for diverse surface modifications and thereby provide avenues for achieving a wide range of chemical selectivity in separation protocols.

To improve the separation characteristics of the polyamide support phase, following formation the support phase can be modified to include chromatography functionality at the surface. More specifically, the polyamide support phase can be modified to include a polymer at the surface. The polymer can carry chromatography functionality for use as a stationary phase during a separation protocol.

Ideally, the polyamide-based support phase can be modified to include the chromatography functionality according to a methodology that can functionalize the entire surface of the support phase, including internal or “hard-to-reach” areas such as along the channel walls of a capillary channel or similar areas of other irregular shaped support phase materials. In addition, the modification technique should be one that will not excessively damage the support phase. For instance, excessive polymer degradation, as may be the case when using UV- or plasma-assisted polymerization techniques should be avoided. In addition, excessive temperature increase should be avoided, as that can lead to undesirable deformation of the support phase, e.g., folding and twisting of individual fibers, which can reduce homogeneity of a column.

Accordingly, in one embodiment, the support phase can be modified by use of a microwave-assisted polymerization process. As utilized herein, the term “microwave” or “microwave spectrum” is intended to refer to electromagnetic radiation within the frequency range of about 300 MHz to about 300 GHz, corresponding to wavelengths of about 1 m to about 1 mm. For example, a microwave source can be similar to those commonly used for industrial and domestic purposes that operate at a frequency of from about 2 GHz to about 5 GHz, e.g., about 2.45 GHz, corresponding to a wavelength of about 12 cm. Microwave-assisted processing of a support phase can offer many advantages including non-contact heating, rapid heating, high levels of temperature homogeneity, selective heating (some materials absorb more microwave radiation than others) and low energy cost. As such, the bulk structure of the support phase can be modified to include the chromatography functionality without damage, e.g., pits, pores, cracks, fiber breakage, deformation, etc.

The addition of a polymer that carries the chromatography functionality to a surface of the support phase can be according to either a “grafting to” methodology or a “grafting from” methodology.” A “grafting to” approach refers to attaching functional polymer chains from reaction solution onto the base surface. In general, however, polymer chains are more thermodynamically favored in the solution phase rather than on the polymer surface. The attached polymer chains increase the steric hindrance for the subsequent grafting. As a result, the “grafting to” approach can be self-limiting with lower grafting densities. The “grafting from” approach refers to “growing” polymer chains from reactive monomers or oligomers in the solution on the support phase surface. In the “grafting from” mechanism, the polymerization is initiated and propagated from the surface, and as such the method can results in some embodiments in greater grafting densities than a “grafting to” method.

According to one embodiment of the modification process, the support phase surface can be functionalized according to a radical grafting polymerization process. In general, a method can include contacting the support phase with a grafting solution that includes polymerizable components, e.g., monomers, oligomers, or other pre-polymers; one or more catalysts; and any other desired components, and polymerizing the polymerizable components at the surface by addition of energy in the microwave range.

The method is applicable to the use of any polymerizable monomer, oligomer, or prepolymer that can provide a formed polymer that includes a chromatography functionality as may be useful in a separation protocol. Exemplary polymerizable materials can include, without limitation, vinylidene chloride, chloroprene, isoprene, dimethylaminoethyl methacrylate, styrene, 1,3-butylene dimethacrylate, hydroxyethyl methacrylate, acrylonitrile, acrylamide, N-vinyl pyridine, glycidyl methacrylate, allyl glycidyl ether, 2-{[2-(allyloxy)ethoxy]methyl}oxirane, N-vinyl caprolactam, N-vinyl pyrrolidone, N-vinyl carbazole, acrylic acid, methacrylic acid, ethyl acrylate, ethyl methacrylate, itaconic acid, isobutylmethacrylate, methyl acrylate, acrylamido-2-methylpropanesulfonic acid, sodium vinyl sulfonate, bis(betachloroethyl) vinyl phosphate, cetyl vinyl ether, divinylether of ethylene glycol, divinyl ether of butanediol, vinyl toluene, vinyl acetate, octadecyl vinylether, dimethylaminopropyl methacrylamide, (3-acrylamidopropyl)trimethylammonium, N-(3-aminopropyl)acrylamide. Moreover, amines of a polymerizable component can be quaternized with benzyl chloride, ethyl iodide, methyl or ethylsulfate. Conversely, monomeric chlorides can be quaternized with tertiary amines to give quaternary ammonium compounds. Some suitable tertiary amines are: n-ethyl morpholine, pyridine, cetyldimethyl pyridine, methylmethacrylate. Of course, a combination of two or more polymerizable components can be grafted to obtain graft copolymers.

Acrylic monomers or prepolymers are encompassed in one embodiment as they are a very versatile family. A large number of acrylic monomers that contain different functional ligands is industrially available and encompassed for use as described herein. Implementation of a modification route as described can provide for the functionalization of a support phase with a variety of acrylic monomers such as (3-acrylamidopropyl)trimethylammonium chloride for strong anion exchange, N-[3-(dimethylamino)propyl]acrylamide for weak anion exchange, 3-allyloxy-2-hydroxy-1-propanesulfonic acid for strong cation exchange and allyl glycidyl ether, e.g., for epoxide coupling chemistry.

The polymer thus formed on the support phase can include any of a variety of ion exchange functionality such as, and without limitation to, carboxylic acid (—COOH), sulfonic acid (—SO₃H), primary amines, secondary amines, tertiary amines and quaternary amines, as well as combinations of ion exchange functionalities.

As mentioned previously, disclosed materials are not limited to ion exchange chromatography and other types of separation are encompassed herein. Table 1, below, provides non-limiting examples of chromatography separation protocols encompassed herein, examples of chromatography functionality suitable for such protocols, and examples of analytes targeted by each type of protocol.

TABLE 1

| Application | Functionality/Ligand | Targeted Analyte |
|---|---|---|
| Strong cation | Sulfonate | proteins, peptide, |
| exchange | | nucleic acids, |
| | | drugs, metal ions |
| Weak cation | Carboxylic acid | proteins, peptide, |
| exchange | | nucleic acids, |
| | | drugs, metal ions |
| Strong anion | Quaternary amine | proteins, peptide, |
| exchange | | nucleic acids, |
| | | drugs, metal ions |
| Weak anion | Primary amine | proteins, peptide, |
| exchange | Secondary amine | nucleic acids, |
| | Tertiary amine | drugs, metal ions |
| Hydrophilic | hydroxyl group or | proteins, peptide, |
| interaction | polyethylene glycol | nucleic acids |
| chromatography | (PEG) | |
| Affinity | Proteins, antibodies, | proteins, |
| chromatography | aptamers, carbohydrates | nucleic acids |
| Immobilized- | iminodiacetic acid or | proteins |
| metal affinity | nitrilotriacetic acid | |
| chromatography | | |
| Metal ion separation | Coordination complexes, | transition metals, |
| and extraction | nitrile, amidoxime | lanthanides, actinides |

In one embodiment, the functionality initially provided by the graft polymerization process can be further modified to provide the final chromatography functionality on the support phase. For instance, a support phase can be modified according to a microwave-assisted graft polymerization process to include a polymer carrying a functionality (e.g., an acrylate, an epoxy, an amine, an azide etc.) and that functionality can then be utilized to bind a different functionality, e.g., an antibody or antibody fragment, aptamer, coordination complex, etc. for use in a separation protocol. The chromatography functionality, which can be directly added via the graft polymerization process or upon further modification of the grafted polymer, can utilize any binding mechanism for any separation protocol. For instance, the chromatography functionality can utilize biological-based recognition and binding protocols. By way of example, the chromatography functionality can be an antibody (or a functional fragment or synthetic equivalent thereof) that can be utilized to specifically bind its antigen in a separation protocol.

The polymerizable component(s) of the graft polymerization modification can be dissolved in a suitable solvent such as dimethylformanide, tetrahydrofurane, tetrahydrofurfuryl alcohol, dimethylsulfoxide, water, methyl, ethyl or isopropyl alcohol, acetone, methyl ethyl ketone and ethyl acetate. Also mixtures of two or more solvents can be used.

Among the catalysts (i.e., free radical initiators) which can be used are (without limitation) ammonium persulfate, potassium persulfate, sodium persulfate, hydrogen peroxide, tert-butylhydroperoxide, ditertbutyl peroxide, benzoyl peroxide, dicumyl peroxide, lauroyl peroxide, tert-butyl perbenzoate and peracetic acid.

The concentration of the monomer in the solution can vary within practically any limits, for example, from about 5% to about 40% by weight of a solution, or from about 7% to about 30% in some embodiments. Depending upon the particular characteristics of the monomer, when large amount of monomer are used in the solution (e.g., about 30% or more for large monomers), large amounts of homopolymer can form in the solution phase and it can be difficult to clean the fibers. This can also lower the permeability of the resultant fiber packed columns. However, extremely low values of monomer, e.g., less than about 5% can result in undesirably low ligand grafting on the support phase.

In general, the amount of initiator needed for polymerization can increase as the percentage of monomer in the reaction solution increases. However, in a concentrated reaction solution, the need to use a substantial amount of initiator can be balanced against the tendency of high quantities of initiator to decrease the molecular weight of the formed polymer. Typically, the weight of the initiator used can be about 20% to about 25% of the weight of the monomer, but the optimal amount can be determined in a given reaction without undue experimentation. Increases in the initiator concentration can lead to greater opportunities for surface activation but also can increase the possibility of terminating the chain propagation, which can result in low degrees of polymerization and low ligand densities. In one embodiment, a solution can include from about 0.1% to about 1% (w/v), or about 0.125% to about 0.5% (w/v) in some embodiments.

The treatment time can be relatively short, for instance from about 2 minutes to about 30 minutes in some embodiments.

Treatment time, monomer concentration, initiator concentration, etc. can be controlled to provide the grafted polymer at the surface of the support phase with a molecular weight that can provide desired ion exchange functionality density without excessive polymer chain length, which could negatively affect a separation protocol. For instance, a grafted polymer can have a number average molecular weight of from about 500 Da to about 100,000 Da, from about 500 Da to about 50,000 Da, or from about 1,000 Da to about 10,000 Da, in some embodiments.

The mechanisms of a microwave assisted initiator decomposition and radical grafting polymerization to a polyamide at a support phase surface can be described in one embodiment by the following reaction scheme, in which the monomer is an acrylic acid monomer and the initiator is potassium persulfate (KPS), an example of which is described in detail in the Example section.

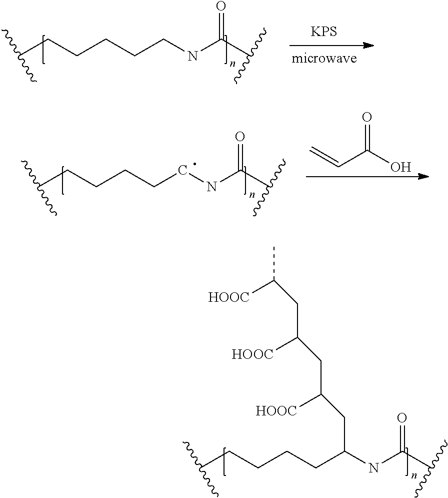

Of course, the above reaction scheme is just one possible embodiment of an acrylic acid graft polymerization, and in other embodiments, the grafting site can be the nitrogen of the amide bond or a combination of carbon and nitrogen. In this particular embodiment, the formed surface can include a high density of carboxylic acid ligands, which can be suitable for use in a weak cation exchange (WCX) protocol.

The separation materials are not limited to acrylic acid based graft polymerization, and as discussed above a support phase can be modified with other materials. For instance, in one embodiment, a support phase can be modified to include a high density of sulfonate groups common to strong cation exchange (SCX) at the surface, one example of which is described in the example section, below, in which the monomer is 2-acrylamideo-2-methylpropane sulfonic acid and the initiator is KPS:

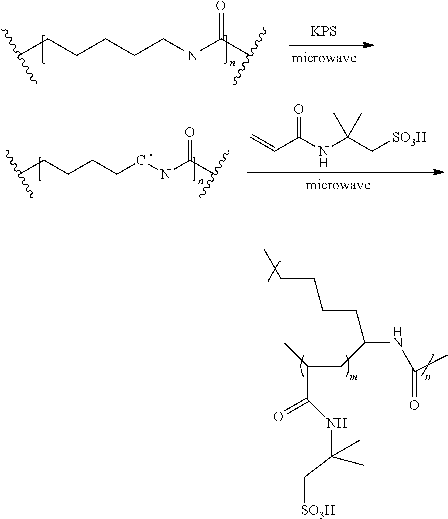

The separation devices can be designed to include the reactive functionality in a wide density range by selection and control of the grafted polymer characteristics (chain length, copolymer formation, etc.). By way of example, the density of the grafted reactive functionality can be present on the support phase in a density of from about 20 μmol per gram of support phase or greater, for instance from about 50 μmol per gram of support phase to about 1000 μmol per gram of support phase in some embodiments. When determined on a surface area basis, the polymer that carries the chromatography functionality can be present on the polyamide support phase at a density of about 500 μmol per m² support phase surface or greater, for instance from about 500 μmol per m² support phase surface to about 1000 μmol per m² support phase surface, for instance based on support phase specific surface area of about 0.7 m² g⁻¹.

In general, the polymeric support phase that carries the chromatography functionality as a stationary phase can be held in a casing such as illustrated in FIG. 1. Casing 22 can be of any material compatible with a solid phase separation protocol. For example, casing 22 can be a glass, ceramic, metallic or polymeric material. In one embodiment, casing 22 can be formed of the same or similar polymeric material as is used to form fibers 20. For instance, casing 22 can be formed of the same base polyamide polymer as is used to form the capillary-channeled fibers 22, though the finished materials may vary somewhat with regard to additives such as clarifiers, nucleating agents, stabilizing agents, other polymers or polymer components, and the like.

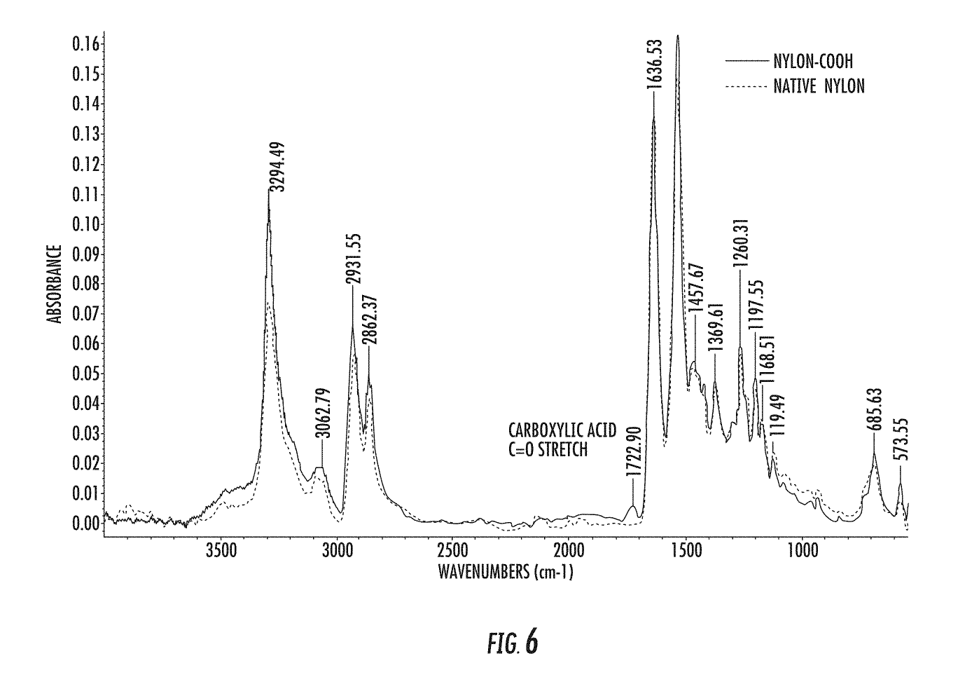

Casing 22 can form the body of a detachable extraction conduit 10 as is illustrated in FIG. 5. A detachable extraction conduit 10 can be used in large or small volume separation protocols. For instance, extraction conduit 10 can be removably attachable to a micropipette tip for use in a small volume micropipette solid phase extraction protocol. Accordingly, an extraction conduit 10 can be of a cross-sectional shape and size so as to be removably attachable to a micropipette tip. Of course, methods for utilizing the surface modified support phase structures are not limited to small volume solid phase extraction protocols, and the surface modified polymeric support phase materials may be utilized in any separation protocol as previously discussed.

An extraction conduit 10 can have an inner diameter suitable for the desired application. For example, the extraction conduit can have an inner diameter from about 0.5 mm to about 5 mm, for instance from about 1 mm to about 3 mm. An extraction conduit need not be circular in cross section, and can describe any cross sectional geometry. The length of a detachable extraction conduit 10 can generally vary depending upon the particularities of the separation to be carried out including volume of the test sample, flow velocity, analyte affinity for the fibers, etc. For example, when considering small volume separation protocols, i.e., less than about 1 mL in volume, an extraction conduit 10, can generally be between about 0.5 cm and about 3 cm in length. In other embodiments, however, an extraction conduit can be longer, for instance up to about 10 cm in length, or even longer in other embodiments, for example when utilizing a large volume sample.

FIG. 5 illustrates one method for forming and using a detachable extraction conduit 10. According to this particular embodiment, a C-CP fiber 20 that has been previously surface modified to include a surface grafted polymer containing chromatography functionality can be fed from a fiber spool 6 to a rotary counter 8. A loop of capillary-channeled polymer fiber 20 containing the desired number of wraps can then be removed from the rotary counter 8 and attached to a monofilament 4. The monofilament 4 can be used to pull the loop of capillary-channeled polymer fibers 20 through the casing 22. The casing 22 containing the fibers 20 can then be trimmed as desired to form a detachable extraction conduit 10.

Disclosed polyamide-based materials can affect highly productive and selective separations in the realm of macromolecule (bio- and synthetic) analytics and as such have wide application in fast protein analytics as well as in the separation of water-soluble polymers. The materials can optionally be utilized to great advantage in a preparative format. Moreover, the formation techniques can provide the ability to create separation columns with a very high level of reproducibility and low cost that can make the columns disposable, if desired.

Beneficially, the ion exchange stationary phase materials can exhibit high loading density. For example, a system can exhibit a protein dynamic loading capacity (DLC) of up to about 15 mg mL⁻¹ bed volume at a linear velocity of about 90 cm min⁻¹, or up to about 10 mg/mL bed volume, up to about 12 mg/mL bed volume, or up to about 15 mg/mL bed volume in some embodiments, for instance from about 8 mg/mL to about 15 mg/mL. In addition, fast (e.g., from about 30 seconds to about 3 min) gradient separations of proteins can be achieved on columns incorporating the modified polyamide support phases.

The separation devices can also have a very high efficiency of mass transfer from the mobile phase to the stationary phase. For instance the devices can have a ratio between the 10% and 50% breakthrough (BT) volumes obtained through frontal analysis of about 0.5 or greater, for instance from about 0.7 to 1, or from about 0.8 to about 0.9, or from about 0.82 to about 0.86 in some embodiments. These values reflect the amount of bound target species (e.g., a proteinaceous macromolecule) at the point when the species concentration of the eluent reaches 10% and 50% of the maximum (solution) concentration. The ratio of the DLC at 10% to the DLC at 50% reflects the sharpness of the breakthrough curve, and is indicative of the high efficiency of mass transfer on the stationary phase.

Disclosed materials can function as the stationary phases in a wide variety of protocols such as 2D stationary phase in comprehensive LC×LC protocols. Moreover, the high level of chemical diversity afforded by the polyimide-based surfaces provides excellent opportunities to affect orthogonality in that dimension. Development of surface modification chemistries to affect higher levels of selectivity is relevant in both analytical scale separations and preparative protocols including, without limitation, LC (both 1D and 2D) and solid phase extraction (SPE) implementation of modified support phase materials.

The present disclosure may be better understood with reference to the Examples set forth below.

### Materials and Methods

### Chemicals and Instrumentation

Unless otherwise specified, chemicals were purchased from commercially available sources and used without further purification. Nylon 6 C-CP fibers were obtained from the Material Science and Engineering department, Clemson University. The denier per filament (DPF) of the fibers employed was 2.67 g per 9000 m, with each fiber having a cross-sectional perimeter of 208 mm. Acrylic acid (99.5%) and potassium persulfate (KPS) (99%) were purchased from Alfa Aesar (Haverhill, Mass.). Activated alumina powder was purchased from Polysciences, Inc. (Warrington, Pa.). 2-acrylamido-2-methylpropanesulfonic acid (AMPS, 99%) and all HPLC solvents were purchased from EMD (Billerica, Mass.). All other chemicals and all proteins were purchased from Sigma-Aldrich (St. Louis, Mo.). Deionized water (DI-H₂O) was secured from a Milli-Q water system.

All chromatograph experiments were performed on a Dionex Ultimate 3000 HPLC system, LPG-3400SD Quaternary pump, and MWD-3000 UV-vis absorbance detector (Thermo Fisher Scientific Inc., Sunnyvale, Calif.). A Rheodyne model 8125 low dispersion injector with either a 3 mL or a 5 mL injection loop was used for protein sample injections. The microwave-assisted polymerizations were performed in a Sunbeam SBM7700W microwave oven, without any modifications of the commercial unit.

### Microwave-Assisted Nylon C-CP Fiber Surface Modification

All DI-H₂O used in the modification reactions was purged with nitrogen for 30 min to remove oxygen prior to use. The acrylic acid was filtered through an activated alumina bed to remove remnant stabilizing agents before use. The modification solution was prepared by dissolving either acrylic acid (2.0 mL, 29 mmol) or the desired amount of AMPS with potassium persulfate (100 mg, 0.37 mmol) in 20 mL DI-H₂O. The native nylon 6 C-CP fibers were removed from the fiber spool and placed on a dying fork. For each modification reaction, 720 fibers of ˜35 cm length were used. The fibers were rinsed with excess amounts of DI-H₂O, methanol, and then DI-H₂O again, to remove any chemical residues left from the fiber extrusion process. The fibers were immersed into the modification solution in a 50 mL beaker and the beaker placed in the microwave oven. Due to the small scale of the reaction, another beaker containing 30 mL water was also placed in the microwave oven as a heat sink for better control and reproducibility of the experiments. The microwave reaction was run at 100 W for 10 min. After reaction, the fibers were removed from the beaker immediately and washed with excess amounts of DI-H₂O until no homopolymer precipitates were visible on the fibers. A control experiment was run with the fibers submerged in neat DI-H₂O instead of in the modification solution to assess any thermal or microwave-induced fiber decomposition.

### Preparation of Fiber Columns

720 native or modified nylon 6 C-CP fibers were pulled through polyether ether ketone tubing (PEEK, 0.762 mm i.d., IDEX Health & Science LLC, Oak Harbor, Wash.) by use of a fishing line. After packing, columns were mounted on the HPLC system and washed to remove any non-covalently bound chemicals (initiators, homopolymers) from the fibers. Once cleaned, the fiber-packed columns were cut to 20 cm length and stored at ambient conditions.

### Characterization of the Modified Fibers

Scanning electron microscope (SEM) images were taken at the Clemson University Electron Microscopy Laboratory, using a Hitachi SU6600 system operating in the variable pressure mode, with a 20 kV accelerating voltage.

Attenuated total reflection-Fourier transform infrared spectroscopy (ATR-FTIR) was performed on a Thermo-Nicolet Magna 550 FITR in the Analytical Testing Lab of the Material Science & Engineering Department, Clemson University. Nylon fiber samples were cleaned with water, methanol and acetone, and the dried under vacuum for 12 hr prior to FTIR analysis.

The ligand densities of native and modified nylon C-CP fibers were determined by acid-base titration. Native and modified fibers were washed in 100 mM HCl solution for 1 min, and then large amounts of DI-H₂O to remove residual HCl from the fibers. The fibers were then washed with acetone to remove water, and dried under mild mechanical vacuum for 20 h at room temperature to remove residual solvent. The dried fibers, were weighed, placed in DI-H₂O, and were titrated with standardized 0.1142 M NaOH solution with phenolphthalein used as the end-point indicator.

The ligand density (D, μmol g−1) was calculated (Eq. (1)):

$$D = \frac{{C}_{NaOH} \times {V}_{NaOH}}{{W}_{Fiber}} \qquad Eq . 1$$

in which C_{NaOH} is the concentration of NaOH solution (μmol mL⁻¹), V_{NaOH} is the volume of NaOH solution used for titration (mL), W_{Fiber} is the weight of the fiber being titrated (g).

The hydrodynamic permeability of the fiber columns was determined by running potassium phosphate buffer (20 mM, pH=6.5) in the columns at different flow rates (0.05-1.5 mL min⁻¹). The backpressure and corresponding mobile phase linear velocity were recorded. Empty tubing was tested under the same conditions as the fiber columns and the backpressures were subtracted from the recorded backpressure of C-CP columns. The permeabilities of C-CP columns were calculated according to the following (Eq. (2)):

$$\frac{Δ P}{L} = \frac{u μ}{{k}_{w}} \qquad Eq . 2$$

in which ΔP is the column pressure drop (Pa), L is the column length in meters, u is the linear velocity of the mobile phase (m s⁻¹), μ is the mobile phase viscosity (Pa s) and k_{w} is the column permeability. The viscosity of 20 mM phosphate buffer was obtained from the literature.

### Liquid Chromatography

All experiments were performed on a Dionex Ultimate 3000 HPLC system. The dynamic loading capacity (DLC) of the columns (column length: 200 mm, i.d. 0.762 mm) was determined through breakthrough (frontal) analysis as known using lysozyme as the test protein. After the column was cleaned and equilibrated with 20 mM potassium phosphate buffer (pH=6.5, designated as buffer A), lysozyme at the chosen concentration in the buffer was introduced to the column. UV absorbance at 280 nm was monitored as a means of detecting breakthrough and quantifying the amount of protein retained on-column. When the UV absorbance of the eluting solution reached a plateau, indicating column saturation, buffer A was then applied on the column to remove non-bound protein. When the absorbance returned to the original baseline, buffer B (1.0 M NaCl in buffer A) was introduced to the column to affect protein elution. The amount of lysozyme retained was determined by integration of the breakthrough curve (equal area method). A blank experiment was performed using an empty PEEK tubing to determine the system hold-up volume/time. The DLC at 10% and 50% breakthrough was calculated at the point that the absorbance of the eluting solution reached 10% and 50% of its maximum absorbance (plateau). While there was very little evidence that it was necessary, columns could be cleaned/regenerated between experiments by passing a 100 mM NaOH solution for 10 min to remove residual, strongly-bound proteins.

The analytical quality of the protein separations was determined using gradient separations (from 100% buffer A to 50% buffer B) of a four-protein mixture. For each separation experiment, 5 mL of a protein mixture containing 0.1-0.25 mg per mL each of myoglobin, α-chymotrypsinogen A, cytochrome C and lysozyme was injected and the chromatogram was recorded at 216 nm. The gradient baseline absorbance was obtained by running the gradient with no protein injected. The absorbance baseline was subtracted from protein separation chromatograms.

### Example 1

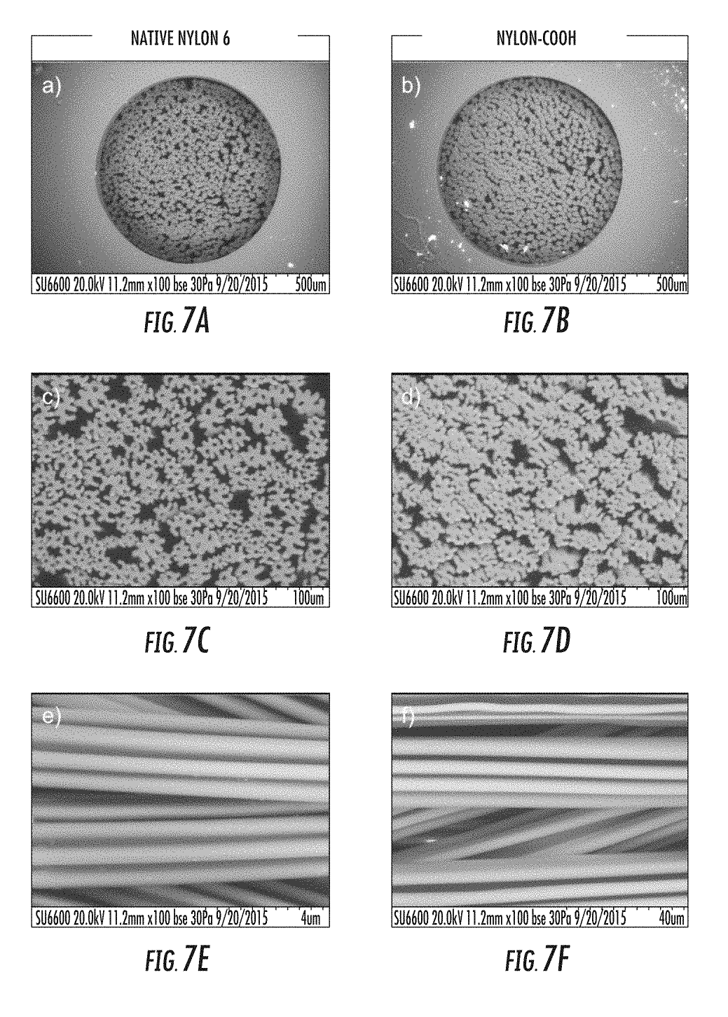

Acrylic acid-functionalized nylon fibers (nylon-COON) were formed and characterized using ATR-FTIR and compared to the native nylon 6 starting material (FIG. 6). Both spectra show virtually identical peaks from the nylon bulk structure: 3294 cm⁻¹ (Amide A, N—H stretch), 3062 cm⁻¹ (NH stretch), 2931 cm⁻¹ and 2862 cm⁻¹ (antisymmetric and symmetric —CH₂— stretch), 1636 cm-1 (Amide I), 1545 cm-1 (Amide II), 1457 cm−1 (CH₂ scissors), 1369 cm⁻¹ (Amide III and CH₂ wagging), 1260 cm⁻¹ (NH bending and C—H stretch), 1197 cm⁻¹ (CH₂ twist-wagging), 1168 cm⁻¹ (CO—NH skeletal motion), 685 cm⁻¹ (out of plane bends of NH), 579 cm⁻¹ (out of plane bends of C═O). The results are consistent with reported values. The peak at 1722.9 cm⁻¹ (C═O stretch) in the Nylon-COON case is consistent with literature reported carbonyl C═O stretch in poly acrylic acid, indicating the presence of those new groups on the nylon-COON fiber surfaces, which were not apparent in the spectrum of the native nylon 6. The fact that the other spectral features are not changed reflects the fact that the polymer backbone was not perturbed by the modification process.

A simple acid/base neutralization titration was applied to specifically quantify the —COON densities. Those determinations yield values of 28±9 μmol g⁻¹ of fiber for the native nylon 6 and 575±7 μmol g⁻¹ for the nylon-COON, or 821±10 μmol m⁻² on a surface area basis.

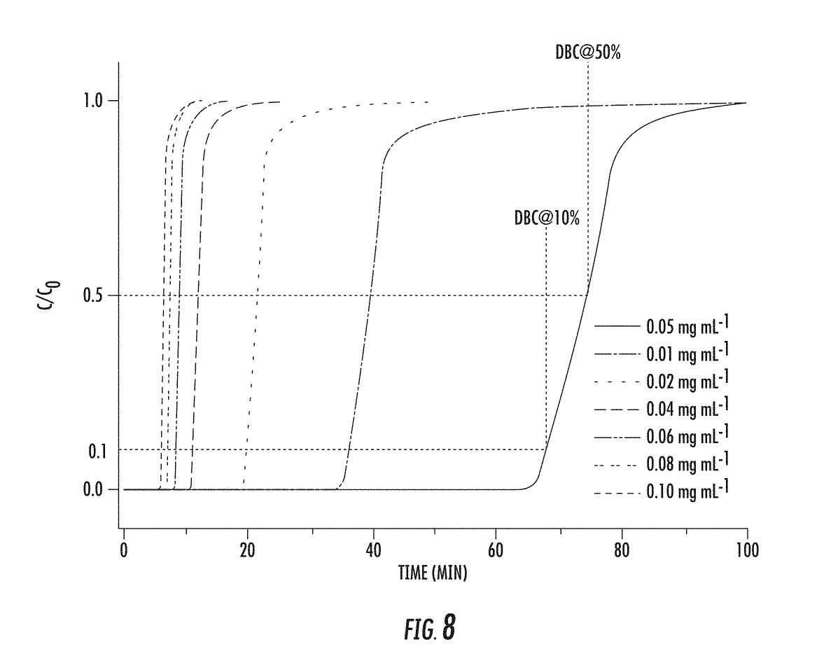

SEM imaging of the fibers provided evidence of any potential macroscopic changes (FIG. 7). The top panels of FIG. 7 (labeled a) and b)) illustrate column cross sections of the native nylon 6 and nylon-COON. As can be seen, the modified fibers are packed more tightly (i.e., their fiber diameters have increased), even though the number of the fibers is the same in both columns (720 fibers). This point is more readily seen in the magnified views in the middle panels labeled c) and d). In the chromatography experiments, the back-pressure of using nylon-COON column was ˜4× higher than using native nylon column (though still comparatively low). Micrographs of control samples of the nylon 6, microwaved in DI-H₂O instead of the modification solution showed no difference in SEM images in comparison to the native fibers. Hence, the increase in fiber thickness following modification appears to be due to the added polyacrylic acid layer from the grafting polymerization. Side-on SEM images (FIG. 7 at the lower panels labeled e) and f)) indicated no macro-damage to the fiber channel structures. These findings support the desired outcome that the grafting polymerization is a non-destructive surface modification method.

The DLC of the native and modified nylon 6 C-CP fiber phases were determined by breakthrough experiments using lysozyme as the model protein. Preliminary assessment of the throughput and yield characteristics of the C-CP fiber phases were done with the same lysozyme/nylon 6 system. Under buffer conditions of pH 6.5, the carboxylic acid on the native nylon (as the end groups) and nylon-COOH fiber phase carry negative charges. Lysozyme has the isoelectric point of ˜11.3 and so has a net positive charge in the buffer, thus it interacts with the native and modified nylon fiber phases via electrostatic interactions. Various concentrations (0.05-1.0 mg mL−1) of lysozyme solutions were loaded onto the fiber columns at a flow rate of 0.4 mL min⁻¹ (Uₒ approximately 29.2 mm s⁻¹). Increasing the ionic strength of the buffer (1.0 M NaCl) eluted the lysozyme from the fiber phases following each loading.

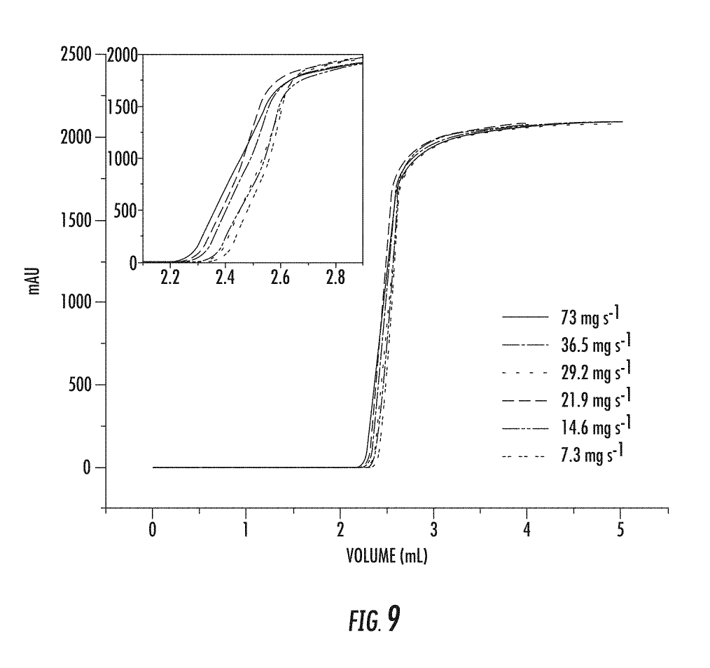

Representative breakthrough curves (UV absorbance at 280 nm) are shown in FIG. 8 As can be seen, as the concentration was increased, breakthrough occurred at shorter times/volumes. Frontal analysis of the breakthrough data in terms of the absolute amounts of protein allows calculation of the dynamic binding capacities presented in Table 2, in terms of the mass of lysozyme per unit fiber mass (mg g⁻¹) and bed volume (mg mL⁻¹). The DLC of nylon fiber phase varied from about 0.4-1 mg mL⁻¹ on the native nylon to about 10-12 mg mL⁻¹ on the nylon-COON.

TABLE 2

| Loading | Native nylon | Native Nylon | Nylon-COOH | | | |
|---|---|---|---|---|---|---|
| Concentration | DLC @50% BT | DLC @50% BT | DLC @50% BT | Nylon-COOH DLC (mg mL⁻¹) | | |
| (mg mL⁻¹) | (mg g⁻¹) | (mg mL⁻¹) | (mg g⁻¹) | @10% BT | @50% BT | 10%/50% Ratio |
| 0.050 | 0.630 ± 0.44 | 0.37 ± 0.26 | 19.1 ± 0.18 | 9.21 ± 0.080 | 10.7 ± 0.10 | 0.86 |
| 0.10 | 1.81 ± 0.020 | 0.70 ± 0.010 | 20.1 ± 0.020 | 9.18 ± 0.020 | 11.2 ± 0.010 | 0.82 |
| 0.20 | 0.870 ± 0.19 | 0.52 ± 0.11 | 20.6 ± 0.010 | 9.44 ± 0.010 | 11.4 ± 0.010 | 0.82 |
| 0.40 | 0.810 ± 0.27 | 0.48 ± 0.16 | 21.1 ± 0.38 | 9.71 ± 0.21 | 11.7 ± 0.21 | 0.83 |
| 0.60 | 1.24 ± 0.47 | 0.74 ± 0.28 | 22.2 ± 0.030 | 10.4 ± 0.020 | 12.4 ± 0.020 | 0.84 |
| 0.80 | 0.940 ± 0.67 | 0.56 ± 0.40 | 22.3 ± 0.040 | 10.4 ± 0.030 | 12.4 ± 0.020 | 0.84 |
| 1.0 | 1.66 ± 0.10 | 0.99 ± 0.060 | 22.6 ± 0.89 | 10.5 ± 0.47 | 12.6 ± 0.50 | 0.84 |

The ratio between the 10% and 50% breakthrough (BT) volumes were obtained through frontal analysis. These ratios on the nylon-COON column ranged from 0.82-0.86, indicative of the high efficiency of mass transfer on the nylon-COON phase. There is no significant change in the 10%/50% BT ratio across the protein feed concentration changes, which would be expected in a diffusion-limited loading situation. This reflects the convective-diffusion driven solute transport that takes place in the C-CP fiber beds.

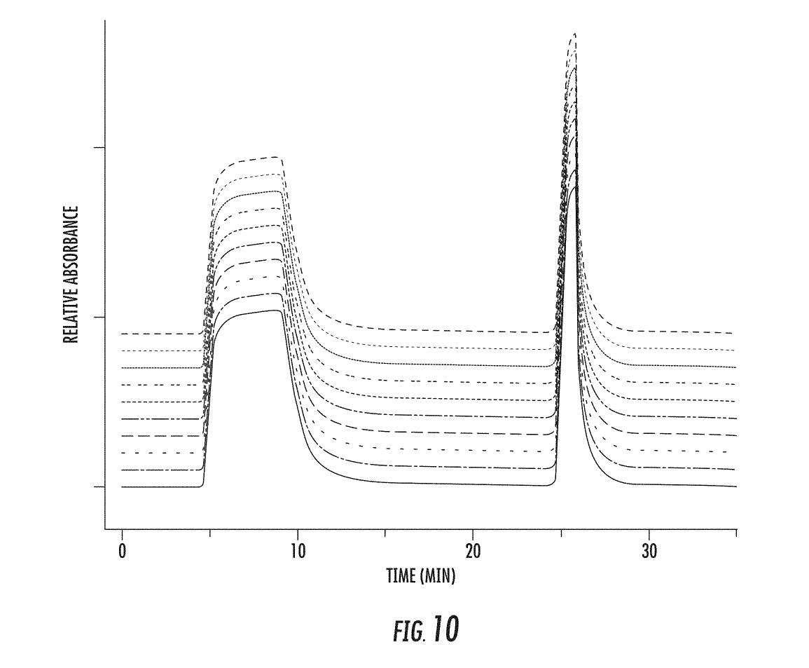

FIG. 9 presents representative lysozyme breakthrough curves on the nylon-COOH column at various flow rates/linear velocities. The lysozyme load concentration was kept at a constant value of 1 mg mL⁻¹. The breakthrough curves were plotted on the basis of the load solution volume to better-reveal any kinetic limitations. As seen in the scale expansion inset, there was a very slight bias in the volume equating to the 50% load, with the slowest application yielding an approximate 4% higher binding capacity than the highest velocity, though the latter occurred at a 10× higher velocity/shorter time scale. Thus, a vast improvement in throughput (T) is realized. It is also interesting to note that the volume displacement at the 10% breakthrough between the different velocities and the 50% level are virtually the same, thus the mass transfer/adsorption kinetics are not sacrificed at the higher linear velocities. Only in the case of the highest linear velocity (73 mm s⁻¹) does it appear that mass transfer limitations are occurring, as the slope of the total breakthrough curve begins to decrease. The negligible difference on DLC at various linear velocities indicates the low mass transfer resistance of the nylon-COON phase at high flow rates, thus fast protein loading/elution can be realized to improve the throughput of protein separations.

Table 3, below, provides several figures of merit for the nylon-COON fiber phase with other commercially-available or literature-reported high permeability/high throughput weak cation exchange phases.

TABLE 3

| | Protein | | |
|---|---|---|---|
| | Binding | | Bed |
| | Capacity | Linear Velocity | Height |
| System | (mg mL⁻¹) | (cm min⁻¹) | (mm) |
| Nylon-COOH | 12 | 44-440 | 200 |
| C-CP fibers | | | |
| Polyacrylate-g rafted | 6 | N/A | |
| polyacrylamide cryogel | | | |
| monolith | | | |
| Methacrylic | 0.065 | <19 | 70 |
| acid-polyethylene | | | |
| glycol diacrylate | | | |
| monolith | | | |
| CLMac ™ CM | 9-11 | 1-10 | 5 |
| monolith | | (recommended) | |
| | | 15 | |
| | | (maximum) | |
| ProSwift WCX-1S | 23 | 6.4-25.6 | 50 |
| monolith | | (recommended) | |
| | | 32 | |
| | | (maximum) | |
| Sartobind ® C | 21 | 2-6 | 4 |
| membrane | | (recommended) | |
| | | 233 | |
| | | maximum | |

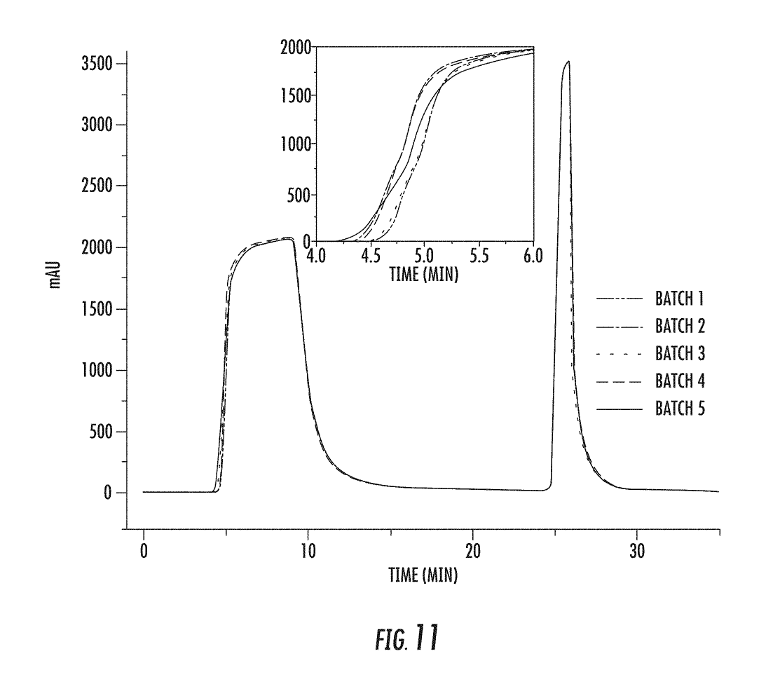

After an initial cleaning using a 100 mM NaOH solution, 10 complete load/elute cycles were executed without any CIP performed in between. (1 mg mL⁻¹ of lysozyme in buffer A was loading into nylon-COON column at 0.5 mL min⁻¹ for 8 min at room temperature (˜20° C.). The column was then washed with buffer A at 0.5 mL min⁻¹ for 15 min prior to elution. Elution was done by feeding buffer B into columns at 0.5 mL min⁻¹ for 7 min.) The subsequent load/elute transients are presented in FIG. 10, stacked from first-to-last from the bottom-to-top. As seen in each case, the nylon-COOH fiber bed is saturated. High consistency is shown, with the load masses (via breakthrough volumes) differing by only 0.2% RSD (n=10) and the recoveries (via the integrated areas under the curve) by only 0.3% RSD (n=10). The same experiment was repeated 6 additional times, with 10 min. 100 mM NaOH CIP exposures between each. Here again, there was no loss in binding capacity or recovery efficiency.

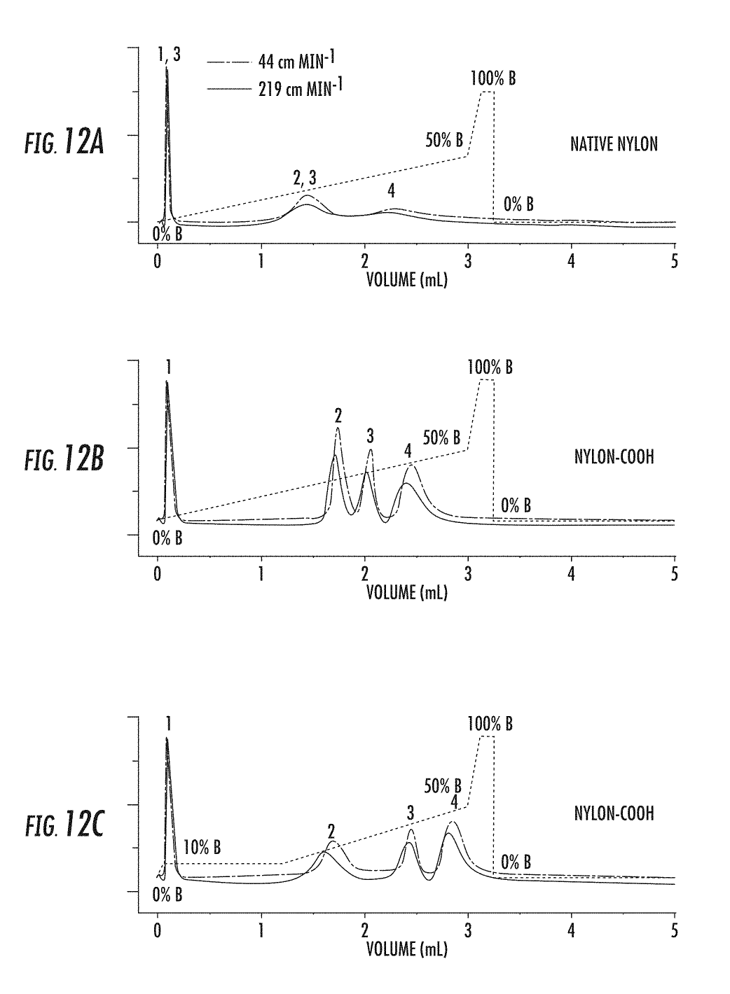

To test the reproducibility of the polymerization process, columns were made once a week for 5 consecutive weeks. Shown in FIG. 11 are the load/elute transients for those 5 columns (1.0 mg mL⁻¹ lysozyme loading concentration at 0.5 mL min⁻¹), directly overlaid to emphasize the quality of the processing. Excellent consistency was attained, with the differences in load masses differing by only 3% RSD (n=5) and the recoveries by only 2% RSD (n=5).

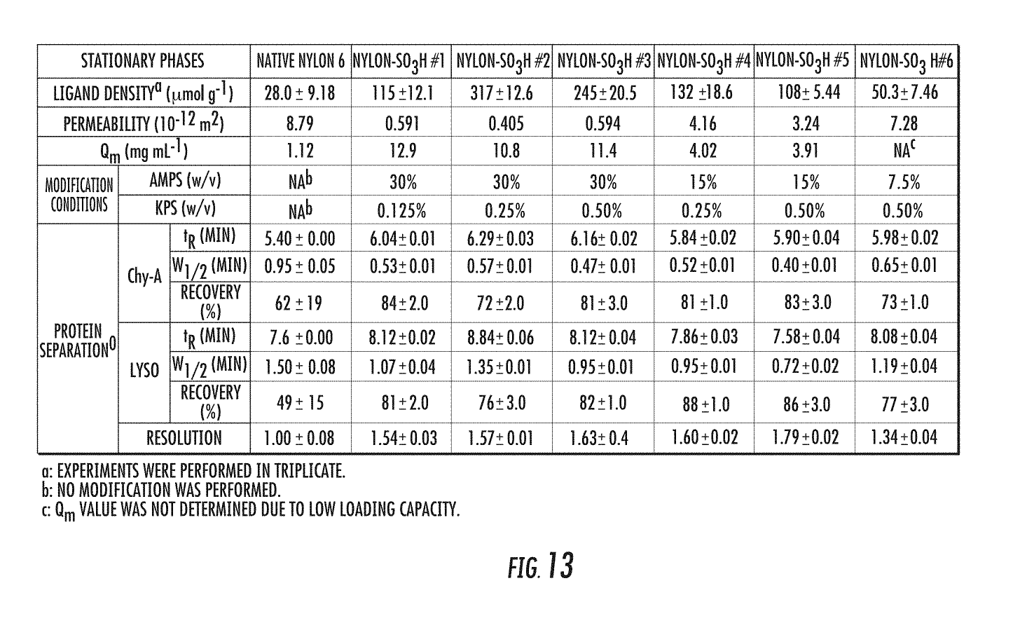

Protein separations were evaluated at linear velocities of 7.3 and 36.5 mm s⁻¹ for a four-protein (myoglobin, α-chymotrypsinogen A, cytochrome C and lysozyme) mixture using a generic NaCl gradient. The chromatograms of the separations are showed in FIG. 12. The dashed lines show the gradient of buffer B used in the separation. Injection volume: 5 μL, Sample concentration: 0.25 mg mL⁻¹ of each protein. Gradients were performed at two flow rates: 0.1 mL min⁻¹ and 0.5 mL min⁻¹. Gradient a (top) and b (middle): 0-3 mL, 0-50% buffer B; 3-3.125 mL, 50-100% buffer B; 3.25 mL, 100% buffer A. Gradient c (bottom): 0-0.25 mL, 0-10% buffer B; 1.2-3 mL, 10-50% buffer B; 3-3.125 mL, 50-100% buffer B; 3.25 mL, 100% buffer A. All chromatograms were recorded by an UV-Vis detector at wavelength of 280 nm and plotted on elution volume basis. All experiments were performed at room temperature (˜20° C.). The top set of chromatograms reflects the protein separations on the native nylon 6 fiber phase. The native nylon has carboxylic acid end groups, however their low density leads to poor retention and broad elution peaks. The cytochrome C component (2) is split into two peaks, the first peak co-eluted with the non-retained myoglobin (1) and the second co-eluting with chymotrypsinogen-A (3) as confirmed by single component injections. Meanwhile, the lysozyme peak (4) exhibits severe tailing and poor recovery. Use of the lower linear velocity (dashed line) provides little significant improvement. The same gradient separations were performed on the nylon-COON column, as in the case of the nylon 6. The four proteins were well separated on the nylon-COON column (middle), with the hydrophilic myoglobin still remaining un-retained. The other proteins show well-behaved responses, with high recoveries. Tuning the gradient results in baseline separation of four proteins (bottom). The increase on the mobile phase linear velocity (5×) did not significantly impair the separation in both (b) and (c). Increases in linear velocity did not diminish the resolution, with a tendency for the proteins to elute at a slightly lower solvent strength. In these instances, the apparent loss of recovery (based on lower absorbance signals) is due to solute dilution per unit time and a time constant bias in the optical detection system.

### Example 2

Native nylon 6 C-CP fibers were modified with 2-acrylamido-2-methylpropanesulfonic acid (AMPS) via the microwave-assisted grafting polymerization to affect a strong cation exchange stationary phase. Various monomer (AMPS) and initiator (KPS) concentrations were used for the grafting polymerization on nylon 6 C-CP fibers.

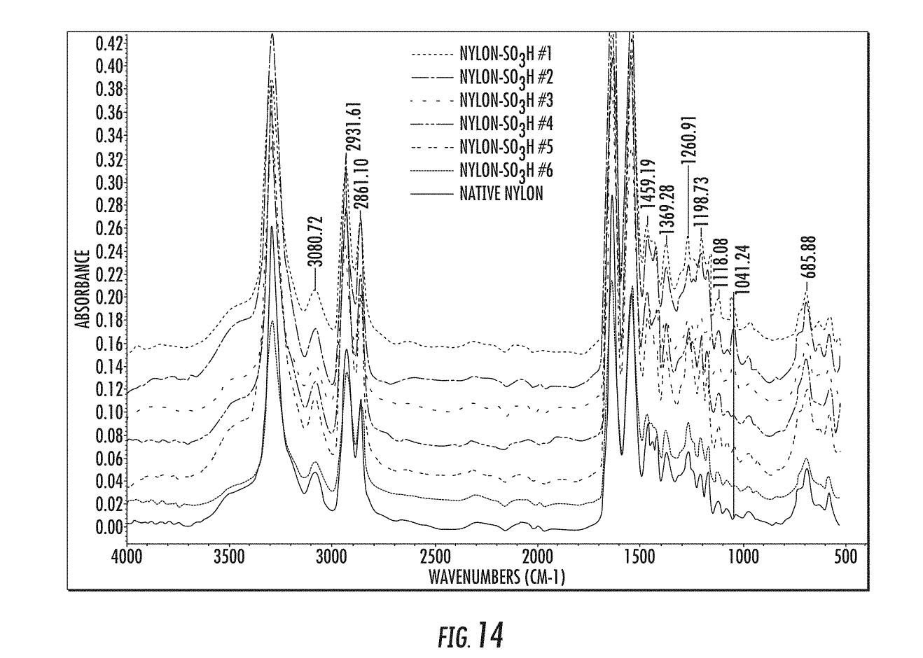

The combinations of reactant concentrations and column characteristics are listed in the table at FIG. 13. Note that the ligand density values reported for native nylon 6 reflect the case where the surface ligand is —COON (Example 1), and not —SO₃. The reaction time, reaction volume and microwave power were applied as described above, with the concentration of AMPS monomer varied from 7.5%-30% (w/v) and the concentrations of the initiator varied from 0.125%-0.5% (w/v). The sulfonic acid ligand densities determined through acid-base titration of the fibers for triplicate reactions are listed in FIG. 13. Without any modification, the native nylon fiber has a ligand density was 28 μmol g⁻¹. The set of tradeoffs in terms of initiator is clearly demonstrated in entries nylon-SO₃H #1-to-3 in the table for the fixed AMPS concentration of 30% (w/v). Doubling of the KPS concentration from 0.125 to 0.25% yielded an ˜2.8× increase the surface ligand density to a maximum value of 317 μmol g⁻¹, while a further doubling of the KPS concentration actually resulted in a reduction in the surface density by ˜25%. Use of lower AMPS concentrations below the highest practical concentration was investigated, yielding the expected results. In comparison to nylon-SO₃H #2, reducing the AMPS concentration by one-half to 15% at the optimum KPS (nylon-SO₃H #4) reduced the determined ligand density by approximately the same proportion. Increasing the KPS concentration from 0.25 to 0.5%, to perhaps compensate for the loss (nylon-SO₃H #5), reduced the ligand concentration as it did at the higher AMPS concentration. A further decrease of the AMPS concentration to 7.5% (nylon-SO₃H #6) led to 50% loss of the ligand density, in comparison to nylon-SO₃H #5. The concentration of AMPS was the limiting factor in the grafting polymerization reactions, while at the highest AMPS concentration (30%) the KPS concentration largely affected the ligand density.

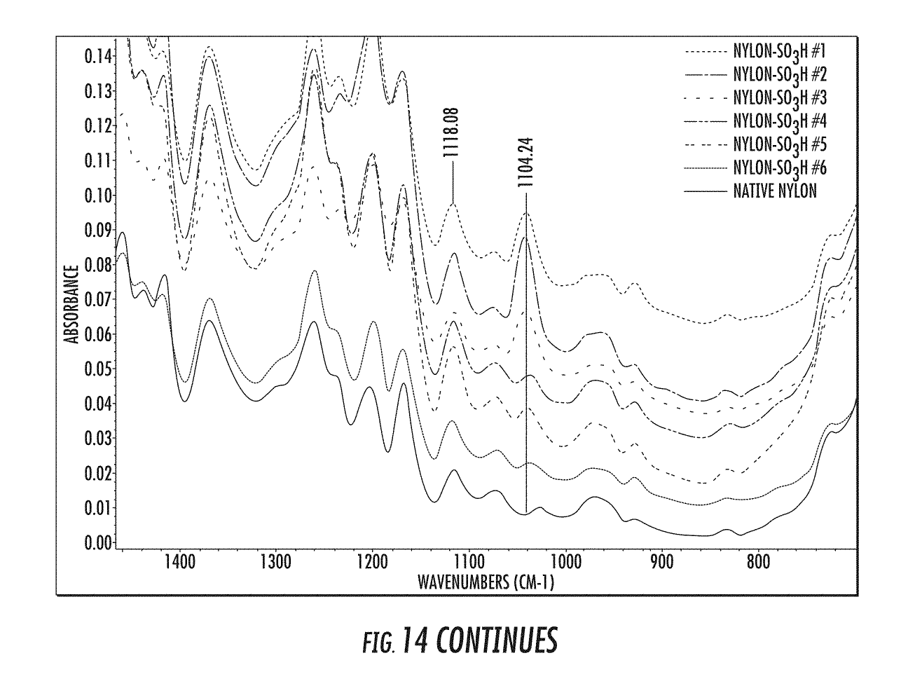

The native nylon 6 and modified fibers were examined by ATR-FTIR to assess the chemical functionality of their surfaces. As shown in FIG. 14 (top), the spectra across the suit of fibers (designated according to the reactant compositions presented in FIG. 13) were virtually identical. The most prominent transitions included: 3295 cm⁻¹ (Amide A, N—H stretch), 3081 cm⁻¹ (NH stretch), 2932 cm⁻¹ and 2861 cm⁻¹ (antisymmetric and symmetric —CH₂— stretch), 1634 cm⁻¹ (Amide I), 1537 cm⁻¹ (Amide II), 1459 cm⁻¹ (CH₂ scissors), 1369 cm⁻¹ (Amide III and CH₂ wagging), 1261 cm⁻¹ (NH bending and C—H stretch), 1199 cm⁻¹ (CH₂ twist-wagging), 686 cm⁻¹ (out of plane bends of NH). The results are consistent with reported values. The sole spectral feature that differed between the native and modified fibers existed at a frequency of 1041 cm⁻¹, corresponding to S═O stretch associated with the —SO₃H ligand present after AMPS modification. Scale expansion around that spectral region (FIG. 14 (bottom)) provided spectroscopic confirmation of the titration-determined ligand densities in FIG. 13. The magnitude of the S═O peak absorbance for the nylon-SO₃H #2 fiber was the highest, followed by those nylon-SO₃H #3 and #1. However, those same transitions show much lower absorbance values in the cases of nylon-SO₃H #4 and #5, even though they have similar densities to those of nylon-SO₃H #1. Thus, high AMPS concentrations yielded long chains, while low concentrations populated many active surface sites and yielded short chains. This understanding agrees with the column backpressure data (below). Thus, while the number of absorbing ligands (based on titration) was similar between treatments #1, #4, and #5, the absorbance for the latter two was much lower due to shorter ligand chains.

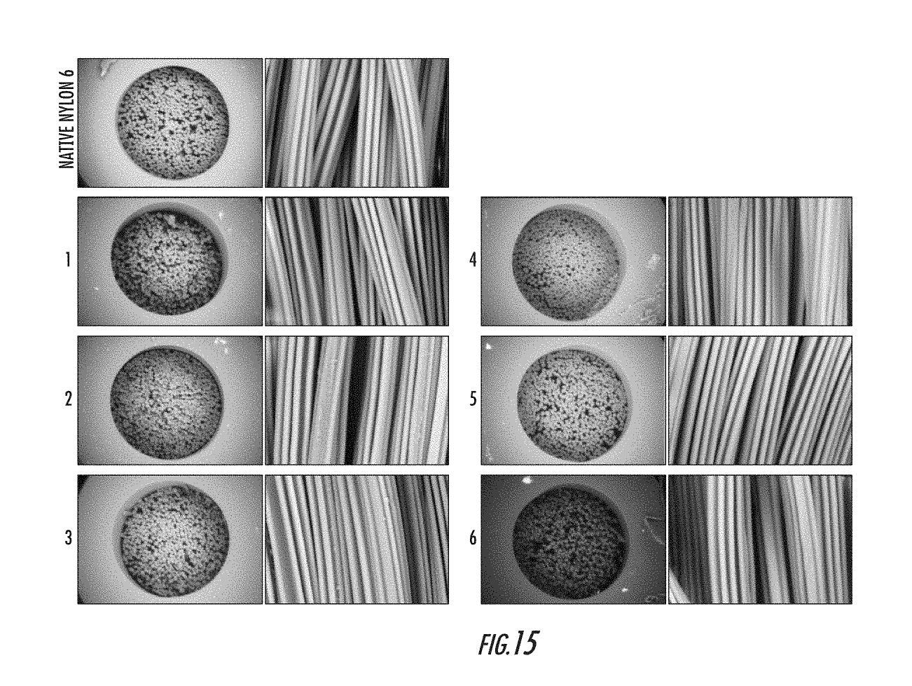

Native and modified nylon fibers and fiber column cross-sections were examined by SEM (FIG. 15). In each column, 720 native or modified fibers were packed in. The cross sectional micrographs reflect a tighter packing of the modified fibers versus the native nylon 6, even though the number of fibers is the same for all cases. The increased packing density after surface modification is due to the increase of fiber diameter. A control experiment was done by microwave heating of native nylon 6 fibers in water instead of the modification solution. The control fiber column sample did not show any increase of packing density or fiber diameter, which indicated the increased thickness was due to the added poly-AMPS overlayer on fiber surface. While not quantitative, the cross sections reflect the fact nylon-SO₃H #2 fiber, having the highest grafted ligand density, had the highest packing density. Indeed, columns composed of fibers having the least number of ligand showed lower packing densities. The side-on micrographs revealed that the C-CP fibers' channel geometries are retained in the modification process.

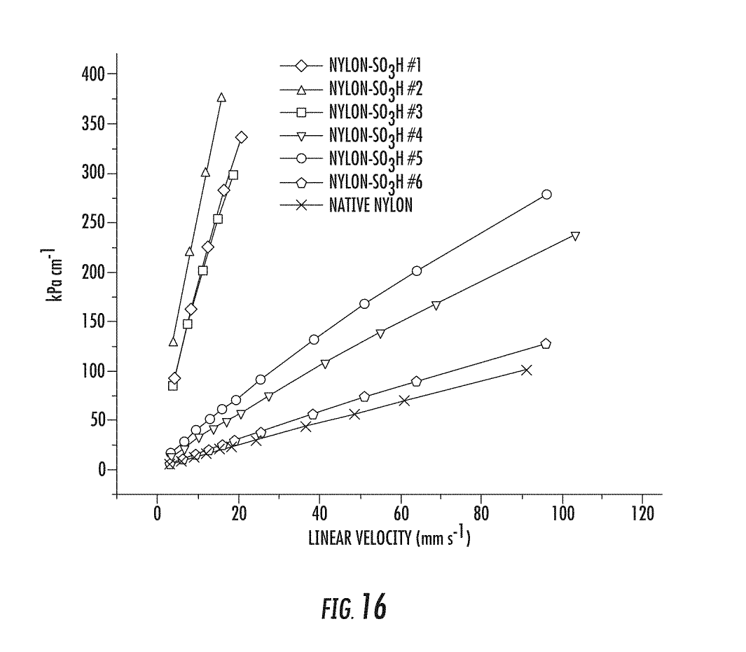

The permeability of nylon-SO₃H columns was determined by pumping mobile phase through columns at different flow rates (linear velocities). The relationship between column pressure drops (kPa cm⁻¹) and mobile phase linear velocity (using 20 mM pH 6.5 potassium phosphate mobile phase) for the native nylon 6 and the modified C-CP fiber column suite are shown in FIG. 16, with the calculated permeability values listed FIG. 13. Colum backpressure was calculated by subtracting the HPLC pump pressure without column installed from the HPLC pump pressure with column installed at different flow rates. The ranges depicted here reflect a maximum operating pressure of ˜7500 kPa for the 20 cm long columns. The linear fits depicted in FIG. 16 indicate negligible stationary phase compression or bed perturbation under the mobile phase linear velocities in the experiments.

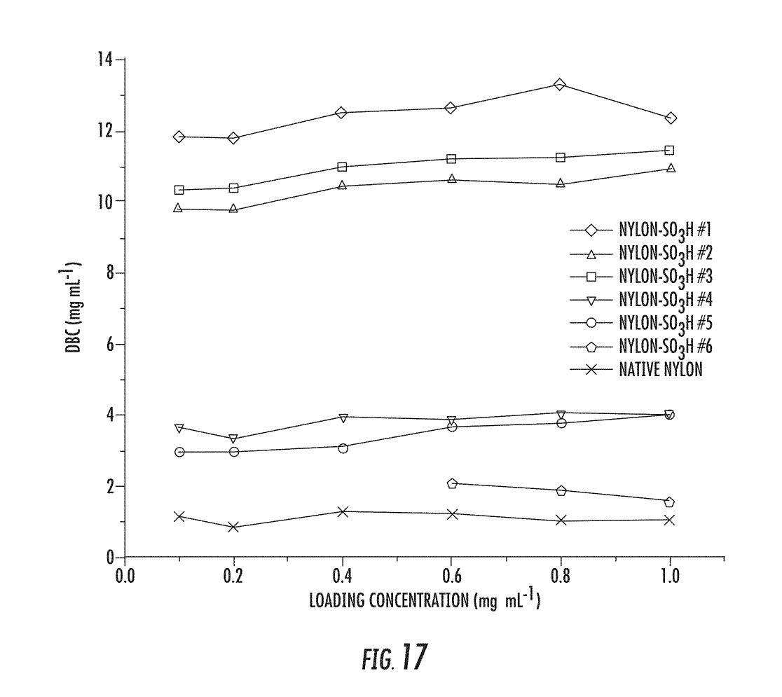

The protein dynamic loading capacity (DLC) of the nylon C-CP fiber columns was determined by breakthrough experiments with lysozyme as the model protein as described above. The determined DLC values are shown in FIG. 17. For all nylon-SO₃H columns, there was virtually no concentration dependence of the dynamic loading capacity on the lysozyme feed concentration. Variations in the feed concentration by 10× (from 0.1 to 1.0 mg mL⁻¹) affected the DLC by ≤0%. This response reflects the fact that the loading is not diffusion-controlled.

Fitting the lysozyme dynamic loading capacity of nylon-SO₃H columns with Langmuir isotherm model resulted in a maximum dynamic binding capacity (Qₘ) of 3.91-12.9 mg mL⁻¹ bed volume. Table 4, below lists several literature-reported SCX phases and commercially available SCX phases.

TABLE 4

| | Protein | | |
|---|---|---|---|
| | binding | | Bed |
| | capacity | Linear velocity | height |
| System | (mg mL⁻¹) | (cm min⁻¹) | (mm) |
| Nylon-SO₃H C-CP fibers | 12.9 | >90 | 200 |
| AMPS grafted | 0.41-1.2 | 2-20 | 80-100 |
| polyacrylamide-based | | | |
| cryogel monolith | | | |
| AMPS grafted | 2.5 | 2 | N/A |
| polyacrylamide-based | | | |
| cryogel monolith | | | |
| AMPS grafted poly(glycidyl | 21.5 | <25 | 150 |
| methacrylate-co-ethylene | | | |
| methacrylate) monoliths | | | |
| 3-Mercaptopropane sulfonic | 8 | 2.55 | 130 |
| acid modified 2-hydroxy- | | | |
| ethylmethacrylate-glycidyl | | | |
| methacrylate monolith | | | |
| CIMmultus ™ SO3-1 | >20 | 2.75-3.25 | 4.2-410 |
| | | (recommended) | |
| CIMac ™ SO3 | 25-31 | 1-10 | 5 |
| monlith | | (recommended) | |
| | | 15 | |
| | | (maximum) | |
| ProSwift SCX-1S | 13 | 6.4-32 | 50 |
| monolith | | (recommended) | |
| | | 38.4 | |
| | | (maximum) | |
| Sartobind ® Q membrane | 25 | 2-6 | 4 |
| | | (recommended) | |
| | | 233 | |
| | | (maximum) | |

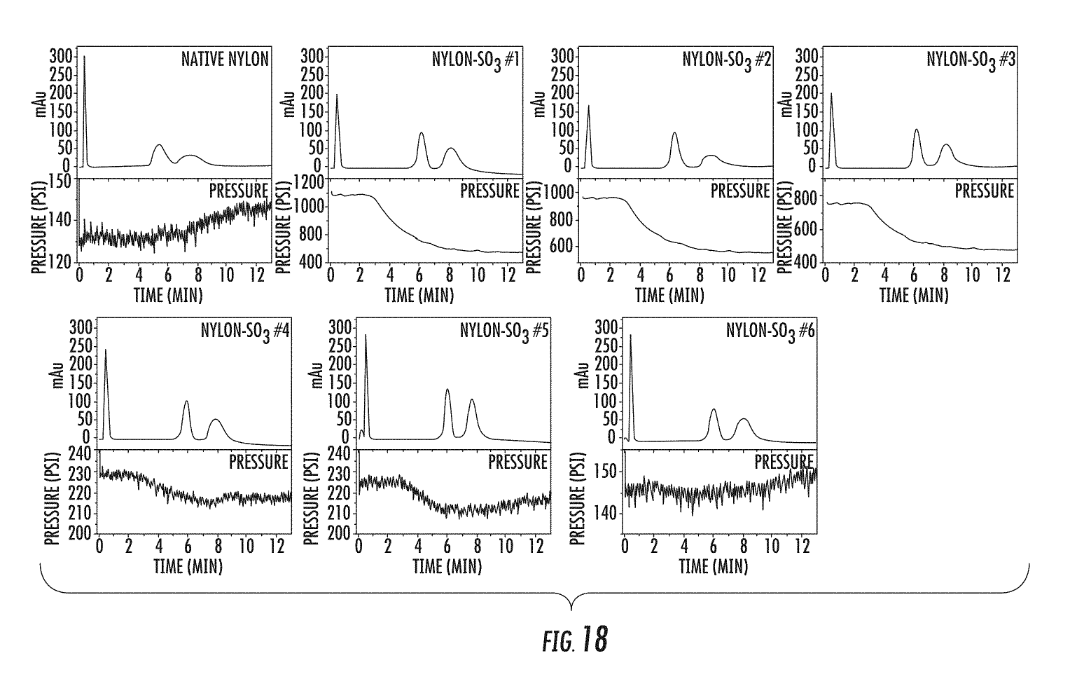

Separations of a three-protein mixture containing myoglobin, α-chymotrypsinogen A and lysozyme were performed on the native nylon 6 and modified nylon-SO₃H columns. A simple NaCl salt gradient was employed. Representative chromatograms for the separation are presented in FIG. 18. Separations were carried out with buffer A (20 mM phosphate, pH 6.5) and buffer B (1 M NaCl in buffer A). The gradient was performed from 0% to 100% buffer B in 10 min. (Gradient: 0 min, 100% buffer A; 10 min: 100% buffer B; 10-11 min, 100% buffer B. Column length: 150 mm, I.D.: 0.762 mm. Flow rate: 0.2 mL min-1, injection volume: 5 μL, sample concentration: 0.1 mg mL-1 of each protein). All chromatograms were subjected to absorbance baseline subtractions based on the same gradients without protein sample injection. As shown, myoglobin was only minimally retained on the columns. This is due to the fact that pI of the protein (6.8-7.3) dictates that it is close to charge neutral in the pH=6.5 buffer. It is also comparatively hydrophilic in nature, and so is not retained to an appreciable extent on the nylon 6 and nylon-SO₃H surfaces. It is clear, though, that myoglobin exhibits peak tailing and broadening on the modified nylon surfaces than on the native nylon surface, reflecting some amount of ionic interactions with the added —SO₃H ion exchange ligands. Additionally, the addition of —SO₃H groups to the nylon surfaces led to enhanced retention (longer retention times) for α-chymotrypsinogen A and lysozyme on all modified columns (except for lysozyme on nylon-SO₃H #6). This was not surprising from the simple point of view that the surface ligand in nylon 6 is low density —COON as opposed to the high density sulfonate groups of the modified fibers.

Nylon-503H #1-#3 showed larger retention time increases while nylon-SO₃H #4-#6 showed less or no retention time increase. The general trend of retention time increase on the modified columns loosely correlates with their different protein binding capacity. Lysozyme was more retained on nylon-SO₃H #6 than on nylon-SO₃H #4 and #5, although nylon-SO₃H #6 shows lower ligand density and protein binding capacity. The larger retention time along with the broader peaks on nylon-SO₃H #6, comparing to nylon-SO₃H #4/#5, suggests a mixed-mode effect of ionic interaction and hydrophobic interactions occurring on nylon-SO₃H #6 surface due to low density of ligand coverage on the native nylon surface. Finally, narrower peaks and less peak tailing were observed after the fiber surface modification. Better peak shapes on the modified columns indicate the increased hydrophilicity of the fiber surfaces after AMPS. As a result, AMPS modification significantly increases the separation resolution of α-chymotrypsinogen A and lysozyme (Rₛ=1.18Δt_{R}/(w_{1/2(1)}+w_{1/2(2)})) on the nylon fiber columns as reported quantitatively in FIG. 13.

As shown in FIG. 18, high columns pressures (750-1100 psi) and large pressure changes during salt gradient (˜400 psi decrease) on nylon-SO3H #1, #2 and #3 indicated the presence of thick layers of poly(AMPS) on the fiber surfaces. This assumption agrees with the large —SO₃H peaks on FTIR spectra of nylon-SO₃H #1, #2 and #3. Thick ligands layers lead to the increased mass transfer resistance thus increase the protein retention. Nylon-SO₃H #1, #4 and #5 had very similar ligand density but largely differed in column pressures. During the modification of nylon-SO₃H #1, the high AMPS concentration (30%) with low initiator concentration (0.125%) led to limited grafting sites on nylon surface but with long grafted poly(AMPS) chains. For nylon-SO₃H #4 and #5, lower AMPS concentration (15%) and higher initiator concentrations (0.25%-0.5%) led to more grafting sites on fiber surfaces with shorter poly(AMPS) chains. This agrees with the differences of the column pressure decreases while larger pressure decrease on nylon-SO₃H #1 but smaller pressure decrease on nylon-SO₃H #4 and #5, indicating the difference on ligand chain length. Due to low ligand density, nylon-SO₃H #6 showed negligible column pressure change during salt gradient. Native nylon showed column pressure increase (˜10 psi) reflecting the increase of mobile phase viscosity with increased salt concentration.

The protein recovery on the modified nylon fiber columns was determined by injecting α-chymotrypsinogen A and lysozyme samples at non-retaining condition (100% mobile phase B) and the case where no column was present, comparing the integrated peak areas with the chromatographic separations. All columns were washed with 100 mM NaOH, 1 M NaCl solution then with 70% method solution, eliminating any bound protein due to ionic or hydrophobic interactions, prior to recovery determination experiments. 3 μL of 0.1 mg mL⁻¹ protein samples were injected to the columns. The injected amount of protein (0.3 μg) was only 0.03%-0.2% of the total protein binding capacity of the columns, thus trace levels of non-specific binding should be seen if occurring. Generally speaking, the recovery of α-chymotrypsinogen A and lysozyme tracked each other among the individual columns/preparation conditions. Nylon-SO₃H #4 and #5 showed the highest lysozyme recoveries, which probably result from the high density of short chain ligands, which provides good hydrophilic barriers. Columns #1 and #3 showed slight lower lysozyme recovery (81% and 82%) than #4 and #5, and #2 showed slightly lower recovery (76%). The increased length of ligand chains thus impaired protein recovery. In the experiments, severe peak tailing (as reflected in W_{1/2}) of lysozyme was seen on nylon-SO₃H #2, which has the highest ligand density. The protein recovery on nylon-SO₃H #6 was lower than nylon-SO₃H #4 and #5, due to its low ligand density. Native nylon showed the lowest protein recovery of (50-60%) due to potential hydrophobic interactions between proteins and the native nylon. Significant protein recovery change was observed during triplicate experiments on native nylon columns. The first experiment showed 43% lysozyme recovery and 44% α-chymotrypsinogen A recovery. The third experiment showed 63% and 71% for lysozyme and chymotrypsinogen A recovery. The increase reflected the hydrophobic interaction on native nylon surface and the low protein binding capacity of the native nylon fibers.

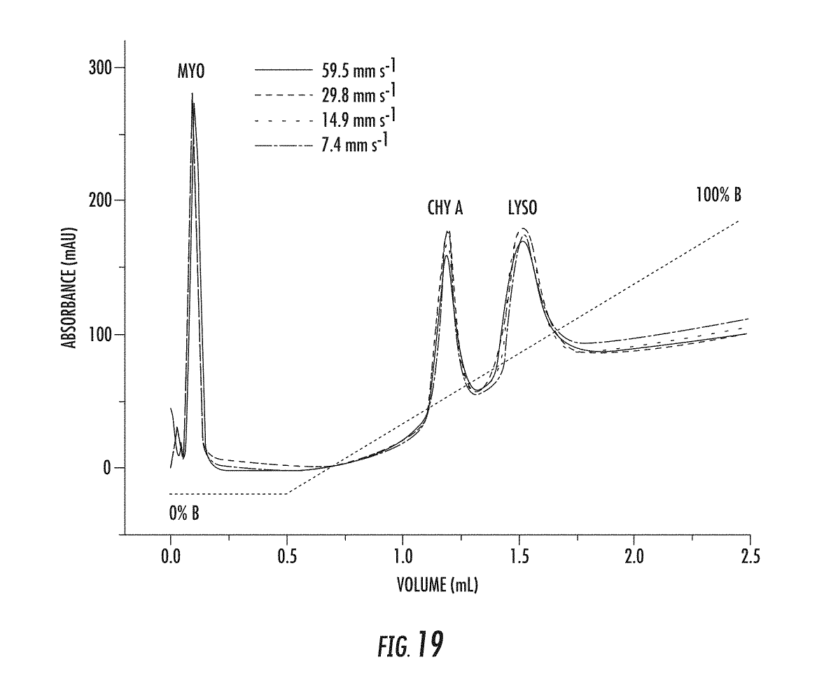

Based on the higher quality of the separation on the nylon-SO₃H #5 column, separations of the three-protein suite at flow rates varying from 0.1 to 0.8 mL min⁻¹ (7.4-59.5 mm s⁻¹) were performed across a gradient of 100% buffer A to 100% buffer B (0 to 1 M NaCl). The gradient rate was held constant across a total volume of 2.0 mL. Plotting of the chromatograms as a function of elution volume (FIG. 19) allowed for an assessment of potential kinetic (predominately van Deemter C-term) limitations. There were no significant changes of elution volume or peak shapes as the flow rate is increased by a factor of 8×.

The consistency of the separation on the nylon-SO₃H #5 column was investigated. Separations of myoglobin, α-chymotrypsinogen A and lysozyme were performed 35 times without column regeneration in between. The variation in the retention time and peak area for α-chymotrypsinogen A were 0.4% RSD and 4% RSD, respectively. The corresponding precision of the more highly retained lysozyme were 0.2% RSD and 5% RSD. In fact, the fluctuations in the elution peak area are due to integration errors from baseline shifts caused by gradient jitter.

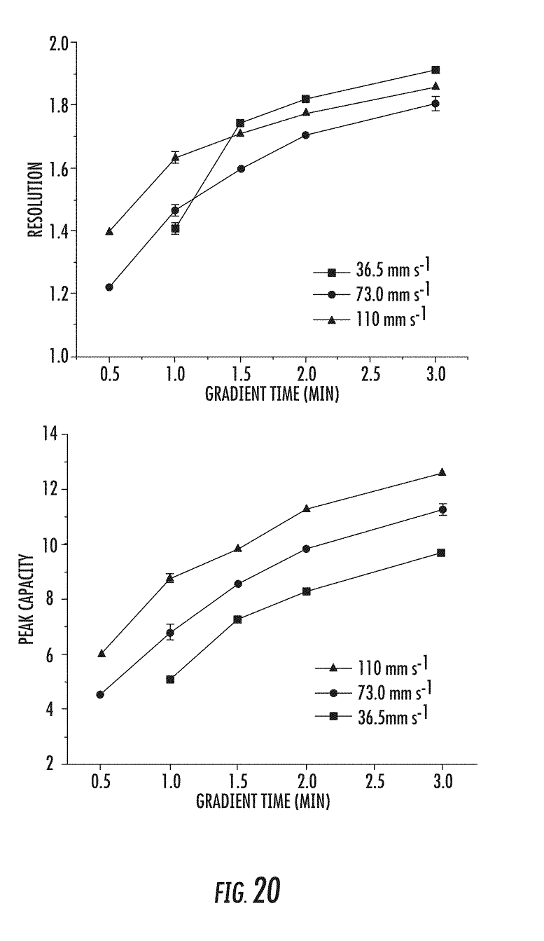

A matrix of high linear velocities and various gradient rates was evaluated for the 3-protein suite, with chymotrypsinogen A and lysozyme chosen as the critical pair. The flow rates used were 0.5, 1.0, and 1.5 mL min⁻¹, corresponding to the linear velocities of 36.5, 73.0 and 110 mm s⁻¹. (the column backpressure at the highest linear velocity remained below 1000 psi for the 20 cm column.) The gradient times studied ranged from 0.5 to 3 min. The responses of the resolution and the peak capacity (PC=t_{G}/w) to changes in gradient times and linear velocities are presented in FIG. 20. For the longer gradient times, the resolution difference among various flow rates was not significant. As gradient time decreases (faster gradients), flow rate has much more profound effects, and high linear velocities that affect the narrowest elution peaks, yield much higher resolution. At the shortest gradient time (0.5 min), the critical pair was not at all resolved at the lowest linear velocity. Thus, under conditions of slow gradients, the differences in component retention times (Δt_{R}) dominate, but for fast gradients, peak width (w) dominates.

As seen in FIG. 20, bottom panel, the ability to operate columns at high linear velocities to affect more narrow peaks is quite clear. As exhibited for many phases, the peak capacity increases with the gradient time. Under the supposition of uniform peak widths, the peak capacity should loosely increase proportionally with t_{G}^{1/2}. This trend is roughly followed in FIG. 20. Moreover, PC values for the nylon-SO₃H #5 C-CP fiber columns increase with mobile phase linear velocity.

While certain embodiments of the disclosed subject matter have been described using specific terms, such description is for illustrative purposes only, and it is to be understood that changes and variations may be made without departing from the spirit or scope of the subject matter.

What is claimed is:

1. A separation apparatus comprising;
a fluid conduit including a first end and a second end;
a support phase disposed within the conduit between the first end and the second end, the support phase comprising a polymeric composition that includes a polyamide at a surface of the support phase;
a polymer grafted to the polyamide at the surface of the support phase, the polymer comprising a chromatography functionality for a separation protocol.

2. The separation apparatus of claim 1, wherein the support phase comprises a plurality of fibers or a capillary-channeled polymer fiber, wherein the fibers are optionally non-porous.

3. The separation apparatus of claim 1, the chromatographic functionality comprising ion exchange functionality, hydrophilic interaction functionality, affinity chromatography functionality, metal ion separation functionality, or combinations thereof.

4. The separation apparatus of claim 1, the chromatographic functionality comprising a carboxylic acid, a sulfonate, a primary amine, a secondary amine, a tertiary amine, a quaternary amine, a hydroxyl, an acetic acid, a nitrile, an amidoxime, an ester, an azide, an alkyne, an epoxide, or combinations thereof.

5. The separation apparatus of claim 1, wherein the fluid conduit is a single use conduit.

6. The separation apparatus of claim 1, wherein the chromatography functionality is present on the support phase in a density of from about 20 μmol per gram of support phase or greater.

7. A method for forming the separation apparatus of claim 1 comprising:
contacting the support phase of claim 1 with a solution, the solution comprising polymerizable monomers or oligomers and a polymerization initiator;
thereafter, contacting the support phase and the solution with energy in the microwave spectrum, thereby initiating radical graft polymerization of the monomers or oligomers to form the polymer grafted at the surface of the polyamide of the support phase.

8. The method of claim 7, wherein the method is carried out at ambient temperature.

9. The method of claim 7, wherein the support phase comprises an irregular cross-sectional shape, the radical graft polymerization being initiated across all of the irregular shaped surface of the support phase that is contacted with the solution.

10. The method of claim 7, wherein the energy in the microwave spectrum is at frequency of from about 2 GHz to about 5 GHz.

11. The method of claim 7, the solution comprising an acrylic acid monomer and/or a sulfonic acid monomer.

12. The method of claim 7, the initiator comprising ammonium persulfate, potassium persulfate, or sodium persulfate.

13. A method for separating a species from a fluid comprising:
moving a fluid through a conduit, the conduit comprising a first end and a second end and a support phase contained between the first end and the second end, the support phase comprising a polymeric composition that includes a polyamide at a surface of the support phase, the conduit further comprising a polymer grafted to the polyamide at the surface of the support phase, the polymer comprising a chromatography functionality for the separation; wherein
upon moving the fluid through the conduit, a species contained in the fluid is retained at the chromatography functionality.

14. The method of claim 13, wherein the species is a macromolecule, for example a proteinaceous compound.

15. The method of claim 13, wherein the species is retained in an amount of from about 8 mg/mL of the support phase to about 12 mg/mL of the support phase and/or wherein the ratio between a 10% breakthrough volume of the species and a 50% breakthrough volume of the species is about 0.5 or greater.
